(19) United States
(12) Patent Application Publication
(10) Pub. No.: US 2022/0241770 A1
(43) Pub. Date: Aug. 4, 2022

(54) FLUID HANDLING DEVICE

(71) Applicant: Enplas Corporation, Saitama (JP)

(72) Inventors: Hayato INOUE, Saitama (JP); Koichi ONO, Saitama (JP); Tomoki NAKAO, Saitama (JP); Shota TAKAMATSU, Saitama (JP); Yasuhiro WATANABE, Saitama (JP)

(73) Assignee: Enplas Corporation, Saitama (JP)

(21) Appl. No.: 17/588,386

(22) Filed: Jan. 31, 2022

(30) Foreign Application Priority Data
Feb. 4, 2021 (JP) 2021-016787

(51) Int. Cl. B01L 3/00; G01N 21/64; B01L 9/00

(52) U.S. Cl. CPC B01L 3/508; G01N 21/6428; B01L 2300/042; G01N 2021/6439; B01L 2300/046; B01L 9/50

(57) ABSTRACT

A fluid handling device includes a board that includes a board body, and a lid that faces the first surface of the board and is disposed so as to form a space between the board and the lid. The lid includes a lid body including a second surface, and a first through hole which allows for injection of a fluid into the space and includes a first opening that opens onto the second surface and a second opening that opens onto a surface other than the second surface. The lid further includes a spacer which is integrally molded with the lid body; alternatively, the board includes a spacer which is integrally molded with the board body.

### CROSS REFERENCE TO RELATED APPLICATIONS

This application is entitled to the benefit of Japanese Patent Application No. 2021-016787, filed on Feb. 4, 2021, the disclosure of which including the specification, drawings and abstract is incorporated herein by reference in its entirety.

### TECHNICAL FIELD

The present invention relates to a fluid handling device.

### BACKGROUND ART

The analysis of DNA base sequence may be used, for example, to test for diseases. A small device capable of analyzing a small amount of sample may be used for the analysis of the base sequence (see, for example, Patent Literature (hereinafter, referred to as PTL) 1).

PTL 1 describes a DNA chip (device) including a substrate, a high reflection region, a low reflection region, and a DNA probe. In the DNA chip described in PTL 1, a target DNA is detected by detecting the fluorescence generated from a fluorescent substance while the target DNA and the fluorescent substance are bound to a DNA probe.

### CITATION LIST

### Patent Literature

PTL 1
Japanese Patent Application Laid-Open No. 2004-317517

### SUMMARY OF INVENTION

### Technical Problem

A possible example of a device for detecting DNA on a board is a device including a board, a spacer forming a side surface of a channel disposed in the board, and a lid forming a ceiling surface of a channel on the spacer. However, in such a device including a board and a lid, and a separate spacer disposed therebetween, it is difficult to form a channel with high accuracy by keeping the joined state between the board and the spacer constant. As a result, the accuracy of the DNA detection result becomes low.

An object of the present invention is to provide a fluid handling device capable of highly accurately forming a space through which a fluid flows by keeping the joined state between a spacer and a lid or board constant.

### Solution to Problem

A fluid handling device of the present invention includes: a board that includes a board body including a first surface; and a lid that faces the first surface of the board and is disposed so as to form a space between the board and the lid, in which

the lid includes a lid body including a second surface that faces the board, and a first through hole that allows for injection of a fluid into the space, the first through hole including a first opening that opens onto the second surface and a second opening that opens onto a surface other than the second surface, in which

the lid further includes a spacer integrally molded with the lid body, the spacer being disposed between the first opening and an outer edge of the second surface in such a way that the spacer is in close contact with the board, or the board includes a spacer integrally molded with the board body, the spacer being disposed between a region facing the first opening and an outer edge of the first surface in such a way that the spacer is in close contact with the lid.

### Advantageous Effects of Invention

The present invention can provide a fluid handling device capable of highly accurately forming a space through which a fluid flows by keeping the joined state between a spacer and a lid or board constant.

### BRIEF DESCRIPTION OF DRAWINGS

FIGS. 1A and 1B illustrate the configuration of a fluid handling device according to embodiment 1 of the present invention;

FIGS. 2A and 2B illustrate the configuration of a board in the fluid handling device according to embodiment 1 of the present invention;

FIGS. 3A and 3B are perspective views illustrating a lid in the fluid handling device according to embodiment 1 of the present invention;

FIGS. 4A, 4B and 4C are diagrams for explaining how to assemble the fluid handling device according to embodiment 1 of the present invention;

FIGS. 5A and 5B illustrate the configuration of a fluid handling device according to a modification of embodiment 1 of the present invention;

FIGS. 6A, 6B and 6C illustrate the configuration of a board in the fluid handling device according to the modification of embodiment 1 of the present invention;

FIGS. 7A and 7B are perspective views illustrating a lid in the fluid handling device according to the modification of embodiment 1 of the present invention;

FIGS. 8A, 8B and 8C illustrate the configuration of a fluid handling device and a magnetic member according to embodiment 2 of the present invention;

FIGS. 9A and 9B are perspective views illustrating a lid with the magnetic member attached thereto in the fluid handling device according to embodiment 2 of the present invention;

FIGS. 10A and 10B illustrate the configuration of a fluid handling device according to a modification of embodiment 2;

FIGS. 11A and 11B are perspective views illustrating a lid with a magnetic member attached thereto in the fluid handling device according to the modification of embodiment 2;

FIGS. 12A and 12B illustrate the configuration of a fluid handling device according to embodiment 3 of the present invention;

FIGS. 13A and 13B are perspective views illustrating a lid in the fluid handling device according to embodiment 3 of the present invention;

FIGS. 14A and 14B are diagrams for explaining how to assemble the fluid handling device according to embodiment 3 of the present invention;

FIGS. 15A and 15B illustrate the configuration of a fluid handling device according to a modification of embodiment 3 of the present invention;

FIGS. 16A, 16B and 16C illustrate the configuration of a board in the fluid handling device according to the modification of embodiment 3 of the present invention;

FIGS. 17A, 17B and 17C illustrate the configuration of a fluid handling device according to embodiment 4 of the present invention;

FIGS. 18A and 18B are perspective views illustrating a lid in the fluid handling device according to embodiment 4 of the present invention;

FIGS. 19A, 19B and 19C illustrate the configuration of a fluid handling device according to a modification of embodiment 4 of the present invention;

FIGS. 20A, 20B and 20C illustrate the configuration of a board in the fluid handling device according to embodiment 4 of the present invention;

FIGS. 21A, 21B and 21C illustrate the configuration of a fluid handling device according to embodiment 5 of the present invention;

FIGS. 22A and 22B are perspective views illustrating a lid in the fluid handling device according to embodiment 5 of the present invention;

FIGS. 23A, 23B and 23C illustrate the configuration of a fluid handling device according to a modification of embodiment 5 of the present invention;

FIGS. 24A and 24B illustrate the configuration of a board in the fluid handling device according to the modification of embodiment 5 of the present invention;

FIGS. 25A, 25B and 25C are perspective views illustrating a lid in the fluid handling device according the modification of embodiment 5 of the present invention;

FIGS. 26A and 26B are perspective views illustrating a board in a fluid handling device according to embodiment 6 of the present invention;

FIGS. 27A and 27B are perspective views illustrating a lid in the fluid handling device according to embodiment 6 of the present invention;

FIGS. 28A, 28B and 28C illustrate the configuration of a fluid handling device according to a modification of embodiment 6 of the present invention;

FIGS. 29A and 29B are perspective views illustrating a board in the fluid handling device according to the modification of embodiment 6 of the present invention; and

FIGS. 30A and 30B are perspective views illustrating a lid in the fluid handling device according to the modification of embodiment 6 of the present invention.

### DESCRIPTION OF EMBODIMENTS

Hereinafter, one or more embodiments of the present invention will be described in detail with reference to the drawings.

### Embodiment 1

Configuration of Fluid Handling Device

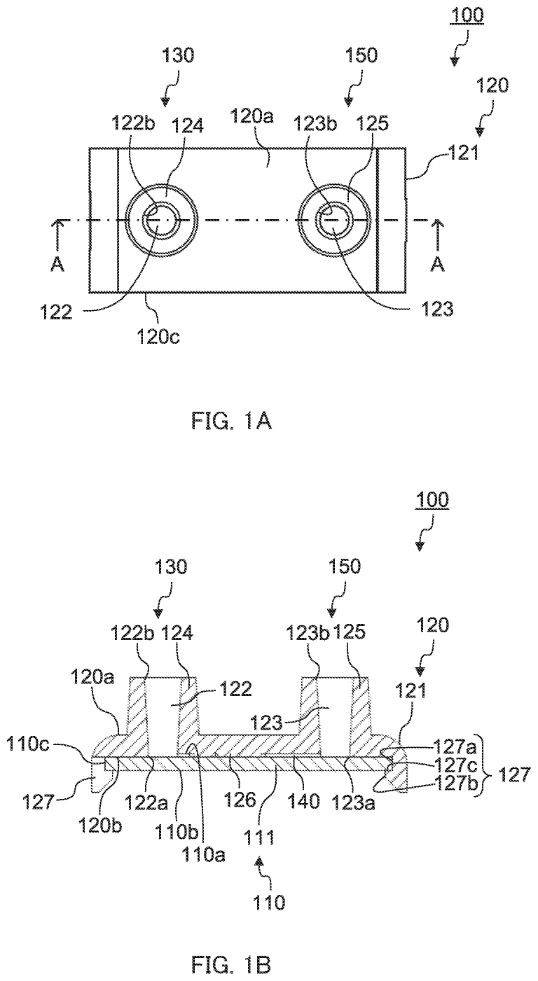

FIGS. 1A and 1B illustrate the configuration of fluid handling device 100 according to embodiment 1 of the present invention. FIG. 1A is a plan view of fluid handling device 100, and FIG. 1B is a cross-sectional view taken along line A-A of FIG. 1A.

As illustrated in FIGS. 1A and 1B, fluid handling device 100 according to the present embodiment is formed of board 110 and lid 120. Fixing board 110 to lid 120 while the board and the lid are in close contact with each other forms inlet 130 for injecting a fluid therein, a channel through which a fluid flows or a chamber for storing a fluid, and outlet 150 for discharging a fluid therefrom. Hereinafter, a channel through which a fluid flows or a chamber for storing a fluid is also referred to as “space 140 or 240 in fluid handling device 100.” Herein, examples of the “fluid” includes liquids and gases.

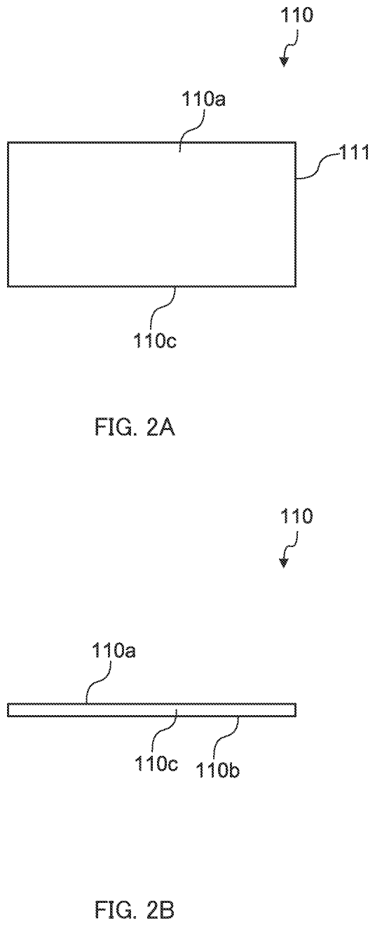

FIGS. 2A and 2B illustrate the configuration of board 110 in fluid handling device 100 according to embodiment 1 of the present invention. FIG. 2A is a plan view of board 110, and FIG. 2B is a side view of board 110.

As illustrated in FIGS. 1A, 1B, 2A and 2B, board 110 is a member that forms the bottom surface of space 140 in fluid handling device 100, in the present embodiment. The external form of board 110 is formed of first front-side surface (hereinafter also referred to as “front surface”) 110a (namely, first surface) disposed to face lid 120, first back-side surface (hereinafter also referred to as “back surface”) 110b located opposite to first front surface 110a, and first outer-side surface (hereinafter also referred to as “outer surface”) 110c which is a side surface disposed at the outer peripheral portion of first front surface 110a and first back surface 110b. Board 110 includes main body of the board (hereinafter also referred to as “board body”) 111. Board 110 may have any shape in plan view. Examples of the shape of board 110 in plan view include polygons, circles, and ellipses. In the present embodiment, the shape of board 110 in plan view is rectangular. Board 110 may have any thickness that allows board 110 to have rigidity sufficient for being held by lid 120. The thickness of board 110 is, for example, in the range of 0.05 to 1.0 mm, for example, 1.0 mm. A reinforcing member (not illustrated) may be joined to the surface—on the side opposite to lid 120—of board 110 for increasing the rigidity. Board 110 may be formed of any type of material. Examples of the types of materials constituting board 110 include silicon, glass, resins, and metals. In the present embodiment, board 110 is a plate-shaped material made of silicon. The silicon may be single crystal silicon or polycrystalline silicon. As board 110 is made of silicon, board 110 has silanol groups on the surface thereof and thus is highly reactive. Fluid handling device 100 including board 110 made of silicon can be used, for example, as a chip for next generation sequencing (NGS). For example, a DNA probe is immobilized on the surface of board 110. The amount of target DNA can be detected by contacting a sample containing a target DNA labeled with a fluorescent substance with the surface of board 110 having the DNA probe immobilized thereon, and detecting fluorescence from the fluorescent substance used for the fluorescent label.

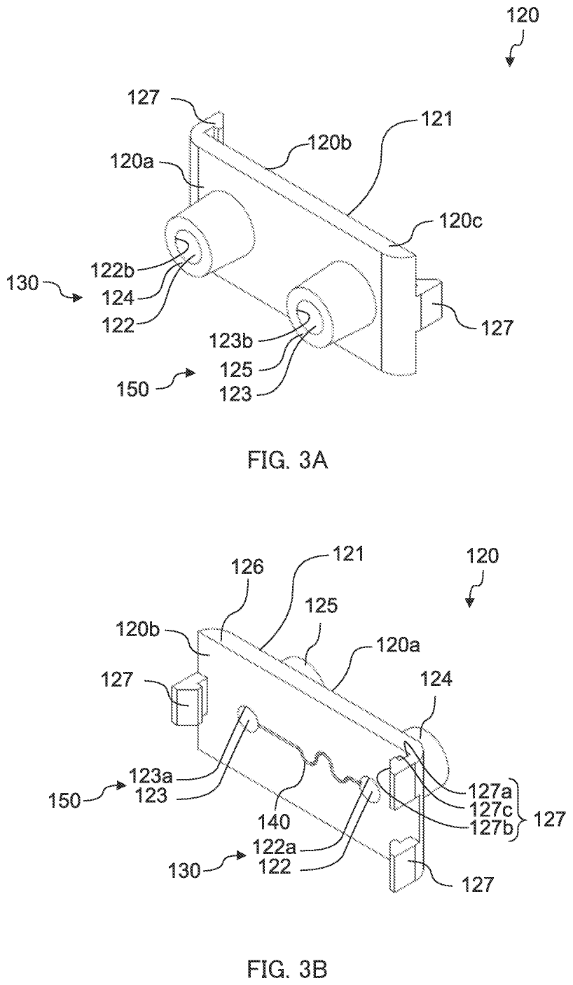

FIGS. 3A and 3B are perspective views illustrating lid 120 in fluid handling device 100 according to embodiment 1 of the present invention. FIG. 3A is a perspective view of lid 120 as viewed from the front side, and FIG. 3B is a perspective view of lid 120 as viewed from the back side.

As illustrated in FIGS. 1A, 1B, 3A and 3B, lid 120 is a member that forms the ceiling surface and the side surface of space 140 in fluid handling device 100, in the present embodiment. Lid 120 is disposed so as to face board 110 and partially form a space between lid 120 and board 110. For using fluid handling device 100 as an NGS chip, any material that allows fluorescence to pass therethrough may be used for lid 120. Examples of the material for lid 120 include resins and glass. Examples of the resins include cycloolefin copolymers (COCs), cycloolefin polymers (COPs), and polymethylmethacrylate resin (PMMA).

Lid 120 includes main body of the lid (hereinafter also referred to as “lid body”) 121, first through hole 122, second through hole 123, first cylinder portion 124, second cylinder portion 125, spacer 126, and at least one claw portion 127.

Lid 120 is formed of second front surface 120a located opposite to the board 110 side, second back surface 120b (namely, second surface) disposed to face board 110, and second outer surface 120c which is a side surface disposed at the outer peripheral portion of second front surface 120a and second back surface 120b.

First cylinder portion 124 and second cylinder portion 125 are disposed on second front surface 120a. In the present embodiment, first cylinder portion 124 and second cylinder portion 125 are disposed along the long side direction of lid 120. In the present embodiment, first cylinder portion 124 is disposed on the left side of second front surface 120a in FIGS. 1A and 1B, and second cylinder portion 125 is disposed on the right side of second front surface 120a in FIGS. 1A and 1B.

First opening 122a of first through hole 122 and third opening 123a of second through hole 123 open onto second back surface 120b. In FIGS. 1A and 1B, first opening 122a opens onto the left side portion of second back surface 120b and third opening 123a opens onto the right side portion of second back surface 120b, in the present embodiment.

Second opening 122b, which is the other opening of first through hole 122, may open onto second front surface 120a, or second outer surface 120c, or open onto the surface via another portion. In the present embodiment, second opening 122b opens onto the top surface of first cylinder portion 124. Similarly, fourth opening 123b, which is the other opening of second through hole 123, may open onto second front surface 120a, or second outer surface 120c, or open onto the surface via another portion. In the present embodiment, fourth opening 123b opens onto the top surface of second cylinder portion 125.

Lid body 121 is a member that forms the ceiling surface of space 140 of fluid handling device 100. The outer shape of lid body 121 when viewed in plan view is not limited. Examples of the outer shape of lid body 121 in plan view include shapes of polygons, circles, and ellipses. In the present embodiment, the outer shape of lid body 121 in plan view is rectangular. The size of lid body 121 in plan view may be larger or smaller than that of board 110. In the present embodiment, the size of lid body 121 in plan view is slightly larger than that of board 110. In addition, in the present embodiment, one or more claw portions 127 are disposed on second back surface 120b of lid body 121.

First through hole 122 allows for injection of a fluid into space 140, and is a part of inlet 130. In the present embodiment, first through hole 122 includes first opening 122a opening onto second back surface 120b and second opening 122b opening onto the top surface of first cylinder portion 124. First through hole 122 may have any diameter that allows for the injection of a fluid. In the present embodiment, the diameter of first through hole 122 is, for example, in the range of 1 to 30 mm. In addition, in the present embodiment, the inner peripheral surface of first through hole 122 is a tapered surface whose diameter decreases from second opening 122b to first opening 122a.

Second through hole 123 allows for discharge of a fluid from space 140, and is a part of outlet 150. In the present embodiment, second through hole 123 includes third opening 123a opening onto second back surface 120b and fourth opening 123b opening onto the top surface of second cylinder portion 125. Second through hole 123 may have any diameter that allows for the discharge of a fluid. In the present embodiment, the diameter of second through hole 123 is, for example, in the range of 1 to 30 mm. In addition, in the present embodiment, the inner peripheral surface of second through hole 123 is a tapered surface whose diameter decreases from fourth opening 123b to third opening 123a.

Spacer 126 is a portion that forms the side surface of space 140 in fluid handling device 100. Spacer 126 is integrally molded with lid body 121, is disposed between the outer edge of second front surface 120a and first opening 122a and third opening 123a (in a region inside the outer edge of second front surface 120a and outside first opening 122a and third opening 123a). Spacer 126 is also configured in such a way that the spacer can be in close contact with board 110. In the present embodiment, spacer 126 is disposed in such a way that space 140 links first opening 122a with third opening 123a. Spacer 126 may have any height so long as space 140 having a desired height is formed between board 110 and lid body 121 when spacer 126 is in close contact with board 110. The height of spacer 126 is, for example, in the range of 5 to 100 μm from the viewpoint of reducing the volume of the fluid. Space 140 linking first opening 122a with third opening 123a may be disposed in such a way that a straight line portion links first opening 122a with third opening 123a, a curved line portion links first opening 122a with third opening 123a, or a straight line portion and a curved line portion link first opening 122a with third opening 123a. In addition, space 140 linking first opening 122a with third opening 123a may be formed in a shape of a liquid storage portion (chamber) having a predetermined capacity.

Claw portion 127 is for holding board 110 by engaging with board 110. Claw portion 127 may have any configuration so long as the above function is exhibited. In the present embodiment, claw portion 127 is disposed on the outer peripheral portion of second back surface 120b in such a way that the claw portion can hold board 110 on the lid 120 side by a snap-fit. Claw portion 127 may be fixed to lid body 121, or may be slidably configured with respect to lid body 121. The number of claw portions 127 is not limited so long as board 110 can be fixed to lid 120. In the present embodiment, the number of claw portions 127 is three. In addition, in the present embodiment, three claw portions 127 are disposed the short sides of lid 120 when the lid is viewed in plan view. In the present embodiment, one claw portion 127 is disposed on one short side and two claw portions 127 are disposed on the other short side.

Claw portion 127 includes fixing portion 127a, guide portion 127b, and holding portion 127c.

Fixing portion 127a defines the position of board 110 in the surface direction (left-right direction in FIG. 1B) in fluid handling device 100. One end of fixing portion 127a is fixed to second back surface 120b of lid body 121, and holding portion 127c is connected to the other end of fixing portion 127a. When viewed from the side, the distance between two fixed portions 127a facing each other is slightly longer than the length of one side of board 110. This configuration can define the position of board 110 with respect to lid 120 in the surface direction when fluid handling device 100 is assembled.

Guide portion 127b guides board 110 when fluid handling device 100 is assembled. Holding portion 127c is connected to one end of guide portion 127b. In a cross section taken along line A-A of FIG. 1A, guide portion 127b is formed in such a way that the guide portion approaches lid body 121 as the distance of the guide portion from the central portion of lid body 121 decreases. This configuration can guide board 110 toward lid body 121 when fluid handling device 100 is assembled.

Holding portion 127c defines the position of board 110 in the vertical direction in fluid handling device 100. Fixing portion 127a is connected to one end of holding portion 127c, and guide portion 127b is connected to the other end of the holding portion. Holding portion 127c is disposed in such a way that the distance between holding portion 127c and lid body 121 is approximately the same as the thickness of board 110. This configuration can define the position of board 110 with respect to lid 120 in the vertical direction when fluid handling device 100 is assembled. A biasing member (not illustrated) that biases board 110 toward lid body 121 may also be provided.

As described above, fluid handling device 100 includes inlet 130, space 140, and outlet 150. Inlet 130, space 140, and outlet 150 are formed by closely contacting board 110 with spacer 126 of lid 120.

Inlet 130 functions to allow for the injection of a fluid into fluid handling device 100. Inlet 130 is a bottomed recess. Inlet 130 is formed of first cylinder portion 124, lid body 121, and a part of board 110.

A fluid is allowed to flow into space 140, or space 140 temporarily stores a fluid. The bottom surface of space 140 is formed of board 110, the side surface of the space is formed of spacer 126, and the ceiling surface of the space is formed of lid 120 (lid body 121).

Outlet 150 functions to allow for the discharge of a fluid in space 140. Outlet 150 is a bottomed recess. Outlet 150 is formed of second cylinder portion 125, lid body 121, and a part of board 110.

After a fluid is injected from inlet 130, for example, applying pressure from inlet 130 or creating a negative pressure on the outlet 150 side allows the fluid of inlet 130 to flow into space 140. The fluid having flowed into space 140 is discharged from outlet 150 to the outside by further applying pressure from inlet 130 or creating a negative pressure on the outlet 150 side.

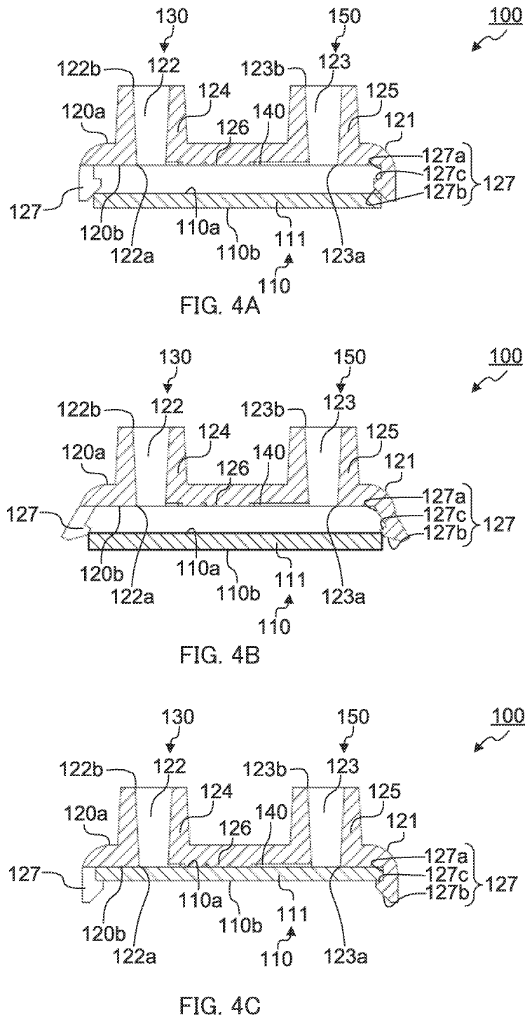

FIGS. 4A to 4C are diagrams for explaining how to assemble fluid handling device 100 according to embodiment 1 of the present invention.

As illustrated in FIG. 4A, board 110 is pressed toward second back surface 120b of lid 120 for attaching board 110 to lid 120. As illustrated in FIG. 4B, when board 110 pushes claw portion 127 toward the outside of lid 120, claw portion 127 is deformed toward the outside. As illustrated in FIG. 4C, when board 110 climbs over holding portion 127c, board 110 is fixed to lid 120, thereby assembling fluid handling device 100. Fluid handling device 100 thus can be assembled as described above.

Modification

The following describes fluid handling system 200 according to a modification of embodiment 1. Fluid handling device 200 according to the modification of embodiment 1 is different from fluid handling device 100 according to embodiment 1 in that board 210 includes spacer 226 in fluid handling device 200. The same components as those of fluid handling device 100 according to embodiment 1 are designated by the same reference numerals, and the description thereof will be omitted. In the following, what is different from fluid handling device 100 according to embodiment 1 will be mainly described.

Configuration of Fluid Handling Device

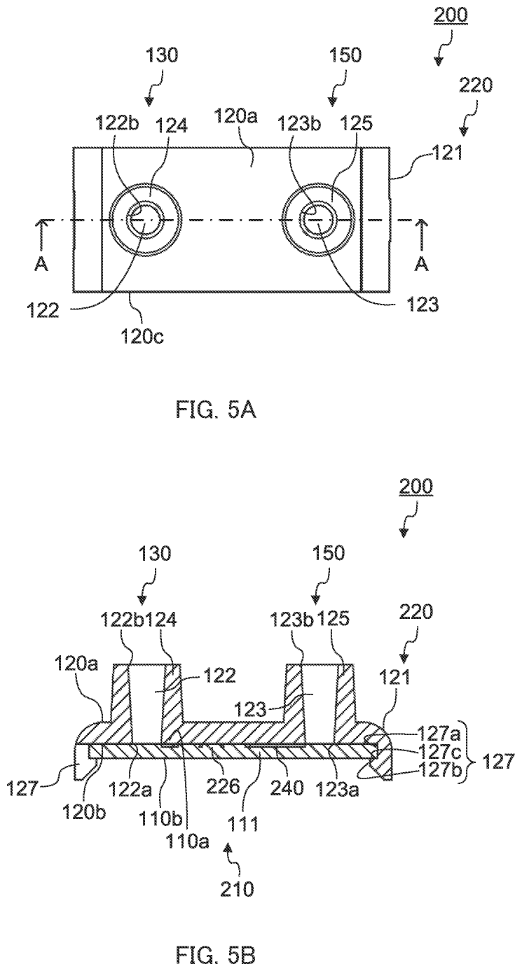

FIGS. 5A and 5B illustrate the configuration of fluid handling device 200 according to the modification of embodiment 1 of the present invention. FIG. 5A is a plan view of fluid handling device 200, and FIG. 5B is a cross-sectional view taken along line A-A of FIG. 5A.

As illustrated in FIGS. 5A and 5B, fluid handling device 200 according to the modification of embodiment 1 of the present invention is formed of board 210 and lid 220.

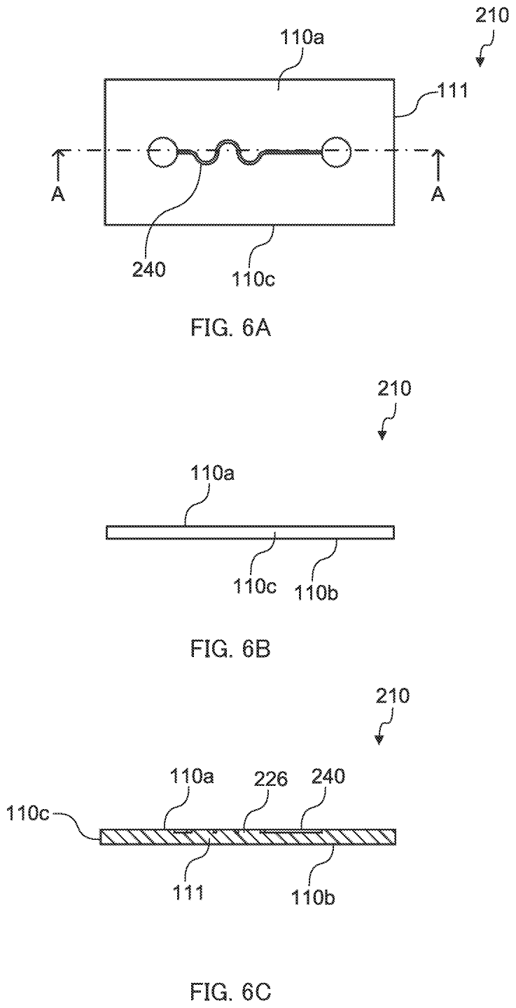

FIGS. 6A to 6C illustrate the configuration of board 210 in fluid handling device 200 according to the modification of embodiment 1 of the present invention. FIG. 6A is a plan view of board 210, FIG. 6B is a side view of board 210, and FIG. 6C is a cross-sectional view taken along line A-A of FIG. 6A.

As illustrated in FIGS. 5A, 5B and 6A to 6C, board 210 in the present modification is a member that forms the bottom surface and the side surface of space 240 in fluid handling device 200. Board 210 includes board body 111 and spacer 226. Spacer 226 is integrally molded with board body 111 of board 210, and is disposed between the outer edge of first front surface 110a and a region facing first opening 122a, and between the outer edge of first front surface 110a and a region facing third opening 123a (in a region inside the outer edge of first front surface 110a and outside the regions facing first opening 122a and third opening 123a) in such a way that spacer 226 is in close contact with lid 220. In the present modification, spacer 226 is disposed in such a way that space 240 links the region facing first opening 122a with the region facing third opening 123a.

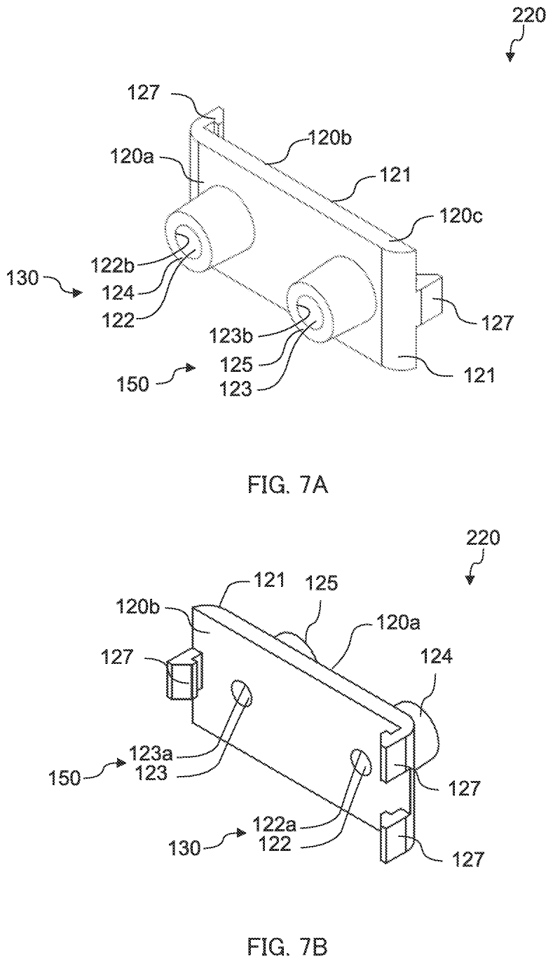

FIGS. 7A and 7B are perspective views illustrating lid 220 in fluid handling device 200 according to the modification of embodiment 1 of the present invention. FIG. 7A is a perspective view of lid 220 as viewed from the front side, and FIG. 7B is a perspective view of lid 220 as viewed from the back side.

As illustrated in FIGS. 7A and 7B, lid 220 in fluid handling device 200 according to the modification of embodiment 1 of the present invention is the same as lid 120 in fluid handling device 100 according to embodiment 1, except that lid 220 does not include spacer 126. In other words, lid 220 includes lid body 121, first through hole 122, second through hole 123, first cylinder portion 124, second cylinder portion 125, and claw portion 127.

Fluid handling device 200 according to the modification of embodiment 1 can be used in the same manner as fluid handling device 100 according to embodiment 1. In addition, fluid handling device 200 according to the modification of embodiment 1 can also be assembled in the same manner (not illustrated) as fluid handling device 100 according to embodiment 1.

Effects

As described above, in the present embodiment, fluid handling device 100 can form space 140 with high accuracy by keeping the joined state between spacer 126 and board 110 constant, and fluid handling device 200 can form space 240 with high accuracy by keeping the joined state between spacer 226 and lid 220 constant. Accordingly, for using fluid handling device 100 as a chip in NGS, for example, the amount of a sample can be kept constant, thereby improving the accuracy of the detection result.

### Embodiment 2

Configuration of Fluid Handling Device

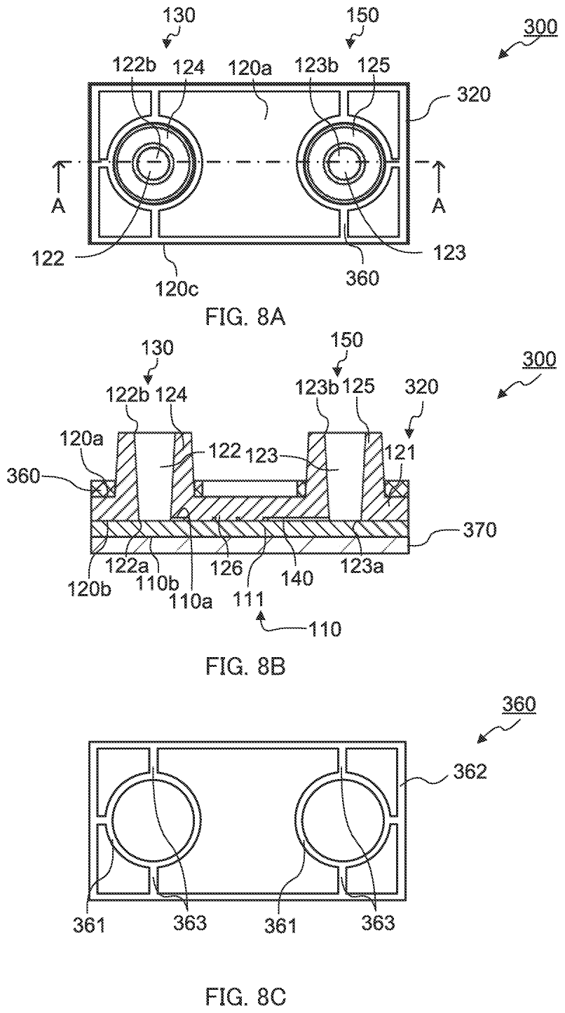

The following describes fluid handling system 300 according to embodiment 2. FIGS. 8A to 8C illustrate the configuration of fluid handling device 300 according to embodiment 2 of the present invention. FIG. 8A is a plan view of fluid handling device 300, FIG. 8B is a cross-sectional view taken along line A-A of FIG. 8A, and FIG. 8C is a plan view of magnetic member 360.

As illustrated in FIGS. 8A to 8C, fluid handling device 300 according to embodiment 2 is formed of board 110, lid 320, and magnetic member 360.

Board 110 is the same as board 110 in embodiment 1, thus the same reference numerals are given and the description thereof will be omitted.

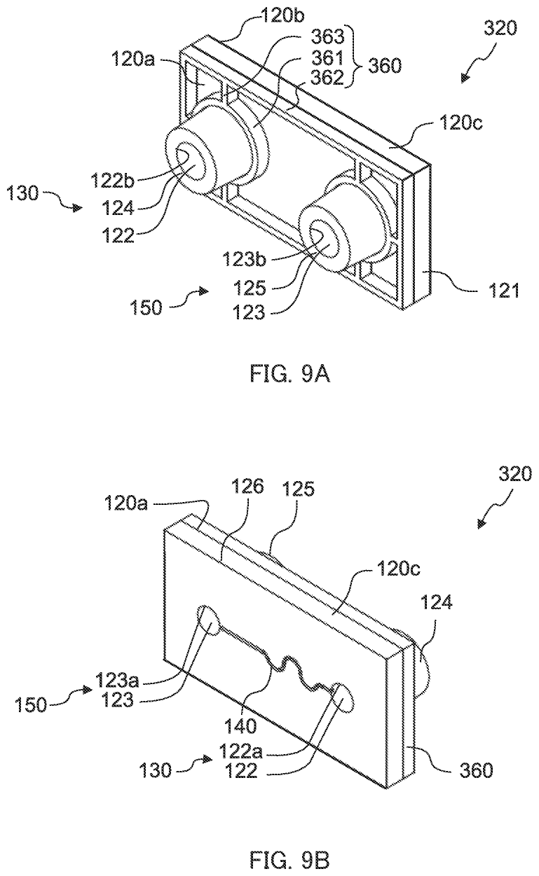

FIGS. 9A and 9B are perspective views illustrating lid 320 with magnetic member 360 attached thereto in fluid handling device 300 according to embodiment 2 of the present invention. FIG. 9A is a perspective view of lid 320 with magnetic member 360 attached thereto as viewed from the front side, and FIG. 9B is a perspective view of lid 320 with magnetic member 360 attached thereto as viewed from the back side.

As illustrated in FIGS. 8A to 8C, 9A, and 9B, lid 320 is the same as lid 120 in embodiment 1, except that lid 320 does not include claw portion 127. In other words, lid 320 includes lid body 121, first through hole 122, second through hole 123, first cylinder portion 124, second cylinder portion 125, and spacer 126.

Magnetic member 360 includes a magnet or a ferromagnet, and is a member for bringing board 110 and spacer 126 of lid 320 into close contact with each other by magnetic force. Examples of the ferromagnet include iron (Fe), cobalt (Co), and nickel (Ni).

Magnetic member 360 brings board 110 and lid 320 into close contact with each other by magnetic force. In the present embodiment, magnetic member 360 includes central portion 361, frame portion 362, and pressing portion 363. Central portion 361 is configured to separately surround first cylinder portion 124 and second cylinder portion 125 of lid 220. Frame portion 362 is an outer edge portion disposed at a position which is closer to the outer edge portion of lid body 121 than that of central portion 361, and which corresponds to the outer edge portion of lid body 121. Pressing portion 363 connects central portion 361 with frame portion 362.

In the present embodiment, board 110, lid 320, and magnetic member 360 are placed on facing member 370 of the main body of the device that uses fluid handling device 300. Facing member 370 includes a magnet or a ferromagnet, and is used to bring board 110 and lid 320 into close contact with each other by a magnetic force, together with magnetic member 360. Examples of the ferromagnet include iron (Fe), cobalt (Co), and nickel (Ni). At least one of magnetic member 360 and facing member 370 includes a magnet. In other words, any one of the following is possible: magnetic member 360 includes a magnet and facing member 370 includes a ferromagnet; magnetic member 360 includes a ferromagnet and facing member 370 includes a magnet; and both magnetic member 360 and facing member 370 include magnets.

Facing member 370 brings board 110 and lid 320 into close contact with each other. Facing member 370 may have any configuration so long as the above function is exhibited. In the present embodiment, the outer shape of facing member 370 in plan view is rectangular as the outer shape of lid 320 in plan view.

Stacking board 110, lid 320, and magnetic member 360 on facing member 370 in this order can bring board 110 and spacer 126 of lid 320 into close contact with each other.

Modification

The following describes fluid handling system 400 according to a modification of embodiment 2. Fluid handling device 400 according to the modification of embodiment 2 is different from fluid handling device 300 of embodiment 2 in that board 210 includes spacer 226 in fluid handling device 400. The same components as those of fluid handling device 300 according to embodiment 2 are designated by the same reference numerals, and the description thereof will be omitted. In the following, what is different from fluid handling device 300 of embodiment 2 will be mainly described.

Configuration of Fluid Handling Device

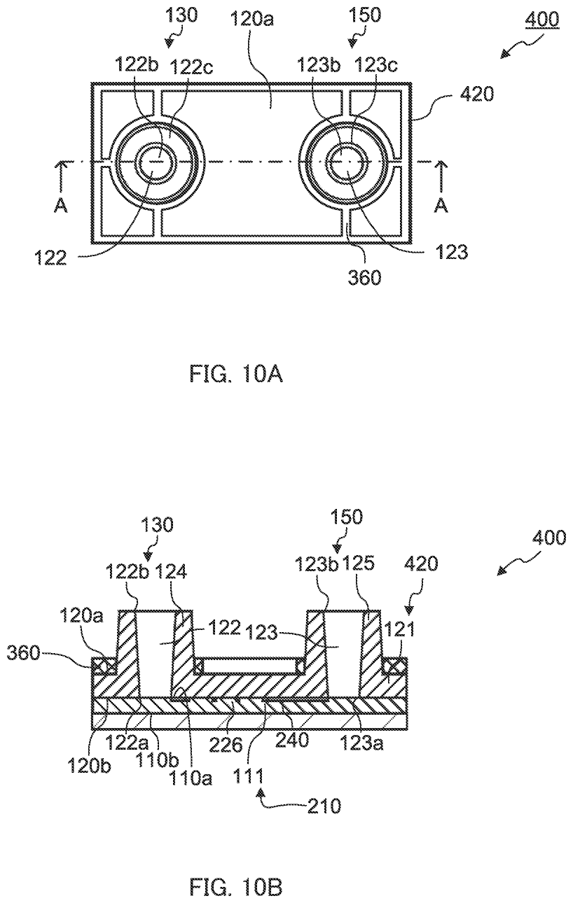

FIGS. 10A and 10B illustrate the configuration of fluid handling device 400 according to the modification of embodiment 2 of the present invention. FIG. 10A is a plan view of fluid handling device 400, and FIG. 10B is a cross-sectional view taken along line A-A of FIG. 10A.

As illustrated in FIGS. 10A and 10B, fluid handling device 400 according to the modification of the present embodiment is formed of board 210 and lid 420.

In the present modification, board 210 is a member that forms the bottom surface and the side surface of space 240 in fluid handling device 400. Board 210 includes board body 111 and spacer 226. Spacer 226 is integrally molded with board body 111 of board 210, and is disposed between the outer edge of first front surface 110a and a region facing first opening 122a, and between the outer edge of first front surface 110a and a region facing third opening 123a in such a way that spacer 226 is in close contact with lid 420. In the present modification, spacer 226 is also disposed in such a way that space 240 links the region facing first opening 122a with the region facing third opening 123a. In other words, board 210 of the present modification is the same as board 210 in fluid handling device 200 according to the modification of the embodiment 1.

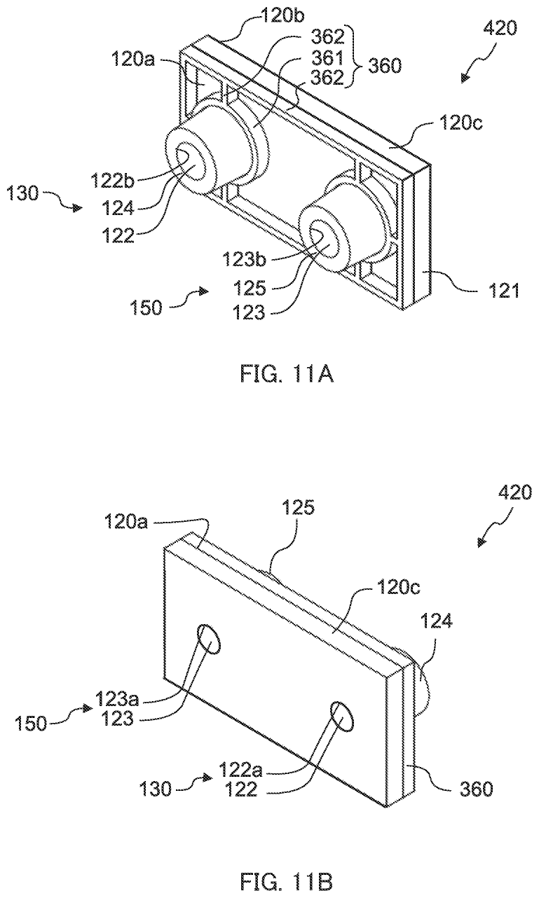

FIGS. 11A and 11B are perspective views illustrating lid 420 with magnetic member 360 attached thereto in fluid handling device 400 according to the modification of embodiment 2. FIG. 11A is a perspective view of lid 420 with magnetic member 360 attached thereto as viewed from the front side, and FIG. 11B is a perspective view of lid 420 with magnetic member 360 attached thereto as viewed from the back side.

As illustrated in FIGS. 11A and 11B, lid 420 includes lid body 121, first through hole 122, second through hole 123, first cylinder portion 124, and second cylinder portion 125. In other words, lid 420 of the present modification is the same as lid 220 in fluid handling device 200 according to the modification of embodiment 1, except that lid 420 does not include spacer 126 or claw portion 127.

Fluid handling device 400 according to the modification of embodiment 2 can be used in the same manner as fluid handling device 300 according to embodiment 2. In addition, fluid handling device 400 according to the modification of embodiment 2 can also be assembled in the same manner (not illustrated) as fluid handling device 300 according to embodiment 2.

In the present embodiment, board 110 or 210, lid 320 or 420, and magnetic member 360 are disposed on facing member 370 of the main body of a device; however, fluid handling devices 300 and 400 may have facing member 370 corresponding to magnetic member 360. In this case, it is highly convenient because the fluid handling devices can be carried while maintaining the close contact state between board 210 and lid 320 or 420. Further, the positions of board 110 and 210 do not shift with respect to the respective positions of lids 320 and 420, thereby further improving the detection accuracy.

Effects

As described above, fluid handling devices 300 and 400 according to the present embodiment can provide the same effects as that of fluid handling device 100 according to embodiment 1.

### Embodiment 3

Configuration of Fluid Handling Device

The following describes fluid handling system 500 according to embodiment 3. Fluid handling device 500 according to embodiment 3 is different from fluid handling device 100 according to embodiment 1 mainly in that spacer 526 forms at least one space 540 in fluid handling device 500. The same components as those of fluid handling device 100 according to embodiment 1 are designated by the same reference numerals, and the description thereof will be omitted. In the following, what is different from fluid handling device 100 according to embodiment 1 will be mainly described.

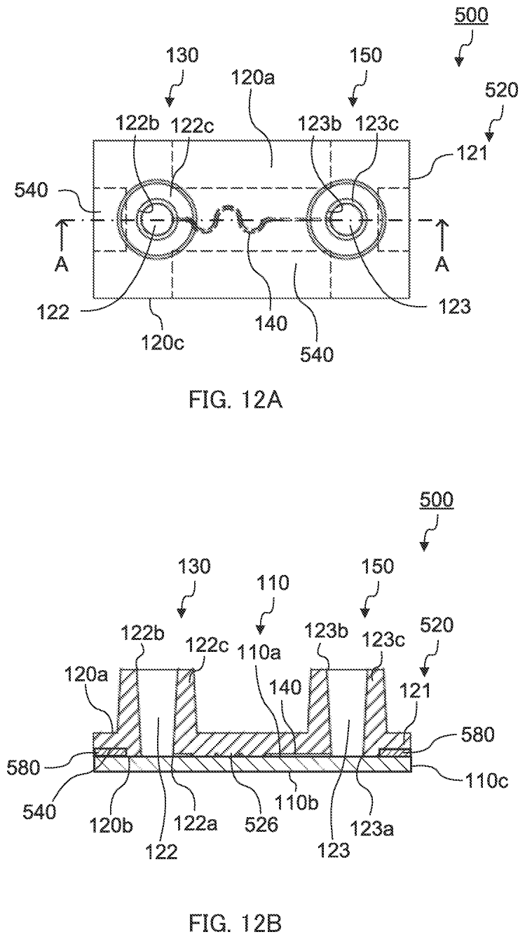

FIGS. 12A and 12B illustrate the configuration of fluid handling device 500 according to embodiment 3 of the present invention. FIG. 12A is a plan view of fluid handling device 500, and FIG. 12B is a cross-sectional view taken along line A-A of FIG. 12A.

As illustrated in FIGS. 12A and 12B, fluid handling device 500 according to embodiment 3 is formed of board 110 and lid 520.

Board 110 is the same as board 110 in embodiment 1, thus the same reference numerals are given and the description thereof will be omitted.

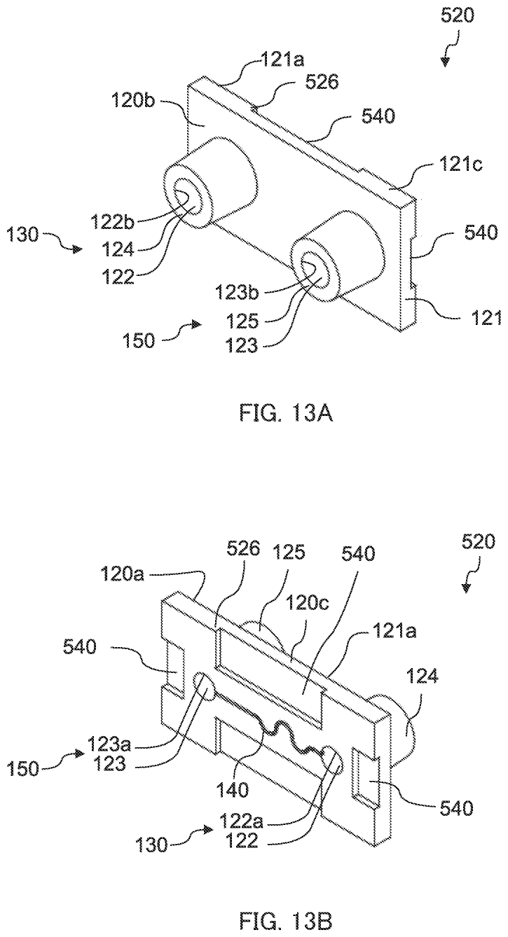

FIGS. 13A and 13B are perspective views illustrating a lid in fluid handling device 500 according to embodiment 3 of the present invention. FIG. 13A is a perspective view of lid 520 as viewed from the front side, and FIG. 13B is a perspective view of lid 520 as viewed from the back side.

As illustrated in FIGS. 12A, 12B, 13A, and 13B, lid 520 includes lid body 121, first through hole 122, second through hole 123, spacer 526, and adhesive layer 580.

Spacer 526 is disposed in such a way that space 140 links first opening 122a with third opening 123a. Spacer 126 also forms spaces 540 open onto the outer edges of lid 520. In the present embodiment, spacer 526 forms five spaces, namely spaces 140 and 540. In the present embodiment, four spaces 540 open at positions corresponding to the four sides of lid 520 when lid 520 is viewed in plan view. Space 540 may have any shape in plan view. The shape of space 540 in plan view may be rectangular, circular, or elliptical. In the present embodiment, the shape of space 540 in plan view is rectangular.

Adhesive layer 580 allows board 110 and lid 520 to adhere to each other. Adhesive layer 580 is disposed in space 540 of spacer 526 so as to link board 110 with lid 520. Adhesive layer 580 may have any configuration so long as the above function is exhibited. Examples of adhesive layer 580 include cured product of adhesives and adhesive sheets. Examples of the adhesives include thermoplastic elastomers such as Zelas (Mitsubishi Chemical Corporation), photocurable adhesives, thermally curable adhesives, and adhesives in combination thereof. Examples of the adhesive sheets include pressure-sensitive adhesives (PSAs) and sheets formed of thermoplastic elastomers or the like.

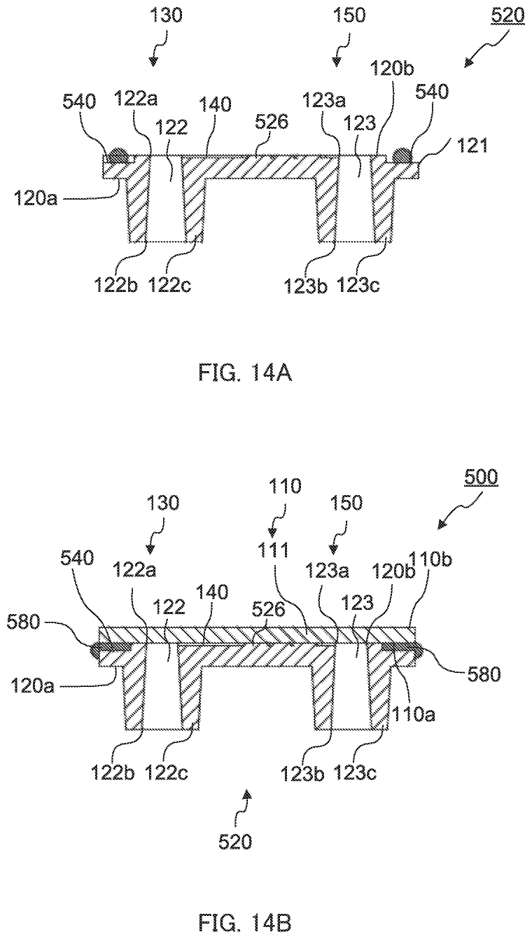

FIGS. 14A and 14B are diagrams for explaining how to assemble fluid handling device 500 according to embodiment 3 of the present invention.

As illustrated in FIG. 14A, an adhesive to become adhesive layer 580 is placed in space 540 of spacer 526 for attaching board 110 to lid 520 for attaching board 110 to lid 520. The volume of the adhesive is equal to or larger than the capacity of the space. Board 110 is then pressed toward second back surface 120b of lid 520. The excess adhesive that is not housed in space 540 protrudes to the outside. This configuration fixes board 110 and lid 520 to each other, thereby assembling fluid handling device 500, as illustrated in FIG. 14B. Fluid handling device 500 thus can be assembled as described above.

Modification

The following describes fluid handling system 600 according to a modification of embodiment 3. Fluid handling device 600 according to the modification of embodiment 3 is different from fluid handling device 500 of embodiment 3 in that board 610 includes spacer 626 in fluid handling device 600. The same components as those of fluid handling device 500 according to embodiment 3 are designated by the same reference numerals, and the description thereof will be omitted. In the following, what is different from fluid handling device 500 of embodiment 3 will be mainly described.

Configuration of Fluid Handling Device

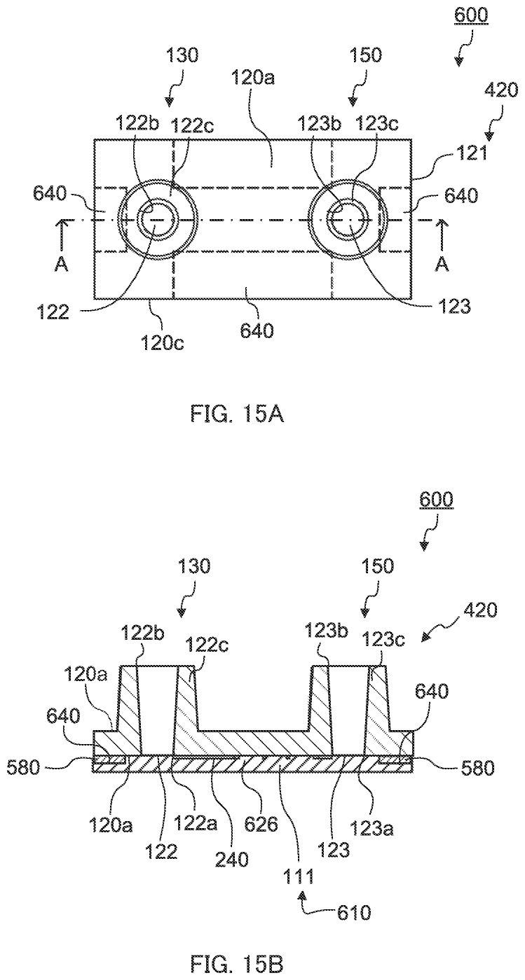

FIGS. 15A and 15B illustrate the configuration of fluid handling device 600 according to the modification of embodiment 3 of the present invention. FIG. 15A is a plan view of fluid handling device 600, and FIG. 15B is a cross-sectional view taken along line A-A of FIG. 15A.

As illustrated in FIGS. 15A and 15B, fluid handling device 600 according to the modification of embodiment 3 of the present invention is formed of board 610 and lid 420.

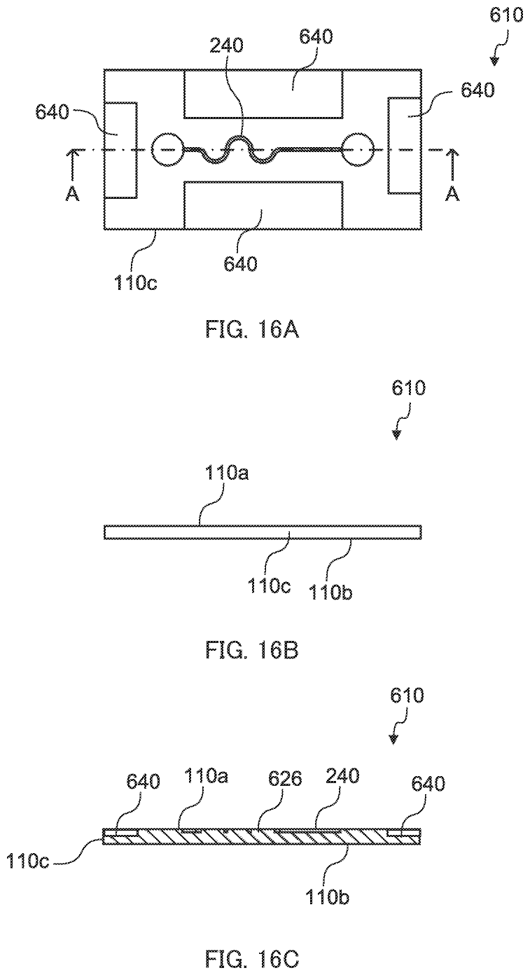

FIGS. 16A to 16C illustrate the configuration of board 610 in fluid handling device 600 according to the modification of embodiment 3 of the present invention. FIG. 16A is a plan view of board 610, FIG. 16B is a side view of board 610, and FIG. 16C is a cross-sectional view taken along line A-A of FIG. 16A.

As illustrated in FIGS. 15A, 15B, and 16A to 16C, board 610 in the present modification is a member that forms the bottom surface and the side surface of space 240 in fluid handling device 600. Board 610 includes board body 111 and spacer 626. Spacer 626 is integrally molded with board body 111 of board 610, and is disposed between the outer edge of first front surface 110a and a region facing first opening 122a, and between the outer edge of first front surface 110a and a region facing third opening 123a in such a way that spacer 626 is in close contact with lid 420. Spacer 626 forms spaces 640 open onto the outer edges of lid 420. In the present embodiment, spacer 626 forms five spaces, namely spaces 240 and 640. In the present embodiment, four spaces 640 open at positions corresponding to the four sides of board 610 when board 610 is viewed in plan view. Space 640 may have any shape in plan view. The shape of space 640 in plan view may be rectangular, circular, or elliptical. In the present embodiment, the shape of space 640 in plan view is rectangular.

Lid 420 includes lid body 121, first through hole 122, second through hole 123, first cylinder portion 124, and second cylinder portion 125. In other words, lid 420 of the present modification is the same as lid 220 in fluid handling device 200 according to the modification of embodiment 1, except that lid 420 does not include spacer 126 or claw portion 127.

Fluid handling device 600 according to the modification of embodiment 3 can be used in the same manner as fluid handling device 100 according to embodiment 1. In addition, fluid handling device 600 according to the modification of embodiment 3 can also be assembled in the same manner (not illustrated) as fluid handling device 500 according to embodiment 3.

Effects

As described above, in the present embodiment, fluid handling device 500 can form spaces 140 and 540 with high accuracy by keeping the joined state between spacer 526 and board 110 constant, and fluid handling device 600 can form spaces 240 and 640 with high accuracy by keeping the joined state between spacer 626 and lid 420 constant. Accordingly, for using fluid handling device 500 or 600 as a chip in NGS, for example, the amount of a sample can be kept constant, thereby improving the accuracy of the detection result. Further, the fluid handling devices include spaces open onto the outer edges, thus the adhesive does not enter spaces 140, 540, 240, and 640, thereby allowing board 110 and 610 to securely adhere to lids 520 and 420, respectively.

### Embodiment 4

Configuration of Fluid Handling Device

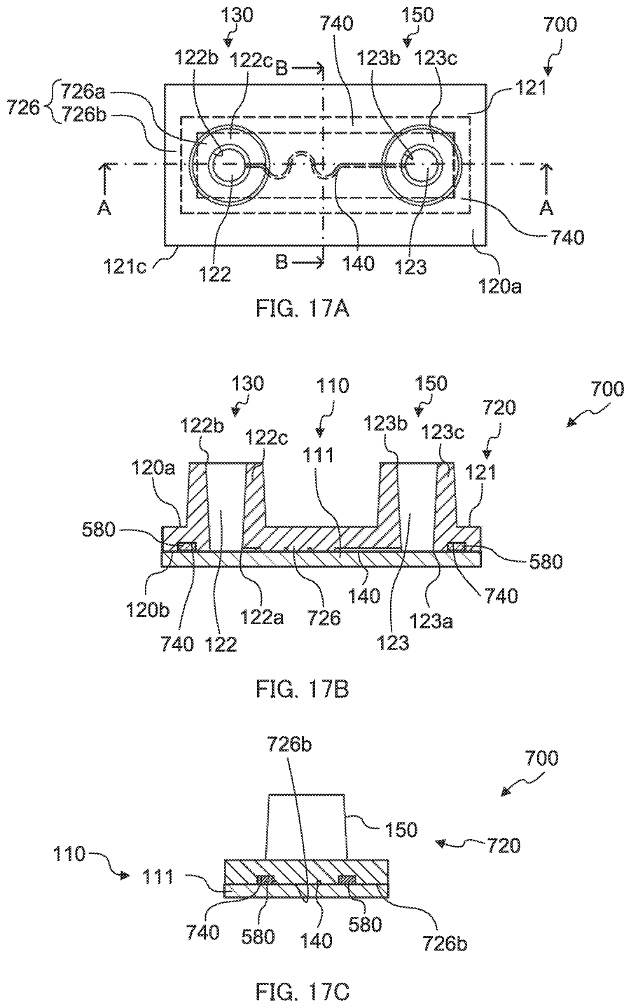

The following describes fluid handling system 700 according to embodiment 4. FIGS. 17A to 17C illustrate the configuration of fluid handling device 700 according to embodiment 4 of the present invention. FIG. 17A is a plan view of fluid handling device 700, FIG. 17B is a cross-sectional view taken along line A-A of FIG. 17A, and FIG. 17C is a cross-sectional view taken along line B-B of FIG. 17A.

As illustrated in FIGS. 17A, 17B, and 17C, fluid handling device 700 according to embodiment 4 is formed of board 110 and lid 720.

Board 110 is the same as board 110 in embodiment 1, thus the same reference numerals are given and the description thereof will be omitted.

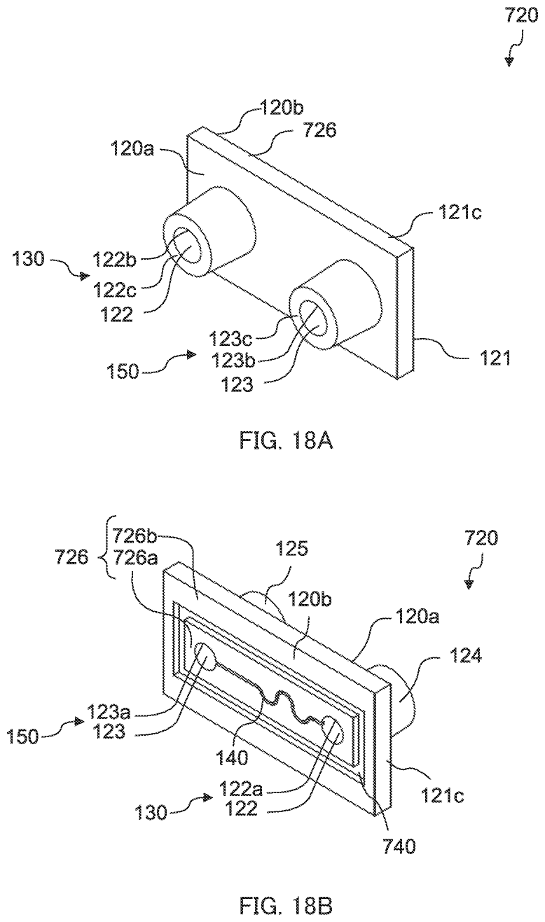

FIGS. 18A and 18B are perspective views illustrating lid 720 in fluid handling device 700 according to embodiment 4 of the present invention. FIG. 18A is a perspective view of lid 720 as viewed from the front side, and FIG. 18B is a perspective view of lid 720 as viewed from the back side.

As illustrated in FIGS. 17A, 17B, 17C, 18A, and 18B, lid 720 includes lid body 121, first through hole 122, second through hole 123, spacer 726, and adhesive layer 580.

Spacer 726 includes first spacer 726a and second spacer 726b. First spacer 726a is formed so as to surround first opening 122a and third opening 123a. In the present embodiment, space 140 is formed from spacer 726 (first spacer 726a) so as to connect first opening 122a with third opening 123a. Second spacer 726b is disposed in such a way that second spacer 726b is closer to the outer edge of first surface 121a than first spacer 726a is, and separated from first spacer 726a. In space 740 between first spacer 726a and second spacer 726b, adhesive layer 580 is disposed so as to link first spacer 726a with second spacer 726b. Adhesive layer 580 is a part of spacer 726.

For attaching board 110 to lid 720, an adhesive (not illustrated) to become adhesive layer 580 is placed in space 740 of spacer 726 in fluid handling device 700 according to the present embodiment. Board 110 is then pressed toward second back surface 120b of lid 720. This configuration fixes board 110 and lid 720 to each other, thereby assembling fluid handling device 700. Fluid handling device 700 thus can be assembled as described above.

Modification

The following describes fluid handling system 800 according to a modification of embodiment 4. Fluid handling device 800 according to the modification of embodiment 4 is different from fluid handling device 100 according to embodiment 1 in that board 810 includes spacer 826 in fluid handling device 800. The same components as those of fluid handling device 100 according to embodiment 1 are designated by the same reference numerals, and the description thereof will be omitted. In the following, what is different from fluid handling device 100 according to embodiment 1 will be mainly described.

Configuration of Fluid Handling Device

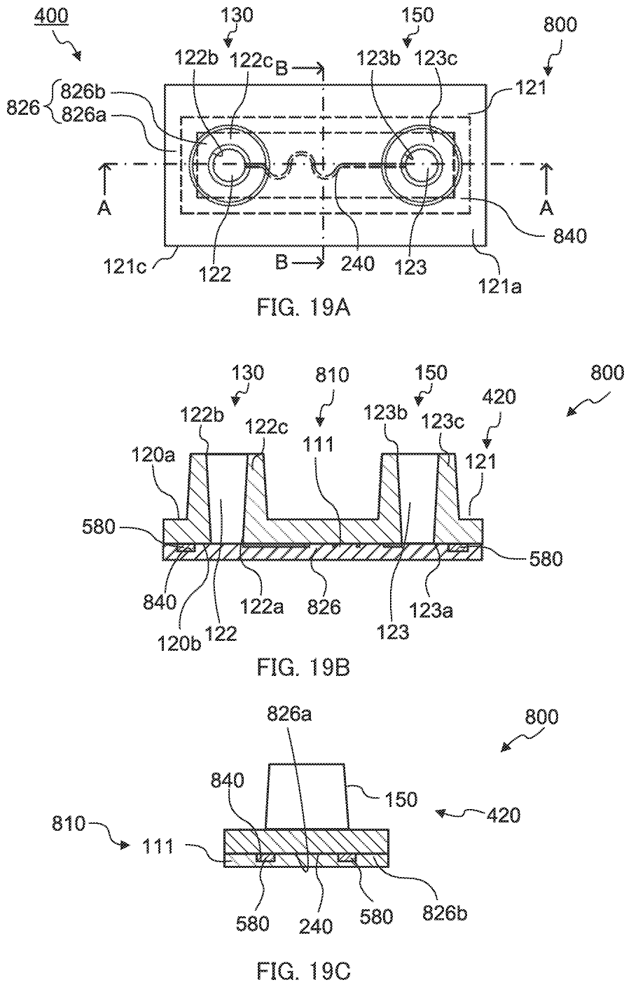

FIGS. 19A to 19C illustrate the configuration of fluid handling device 800 according to the modification of embodiment 4 of the present invention. FIG. 19A is a plan view of fluid handling device 800, FIG. 19B is a cross-sectional view taken along line A-A of FIG. 19A, and FIG. 19C is a cross-sectional view taken along line B-B of FIG. 19A.

As illustrated in FIGS. 19A to 19C, fluid handling device 800 according to the modification of embodiment 4 of the present invention is formed of board 810 and lid 420.

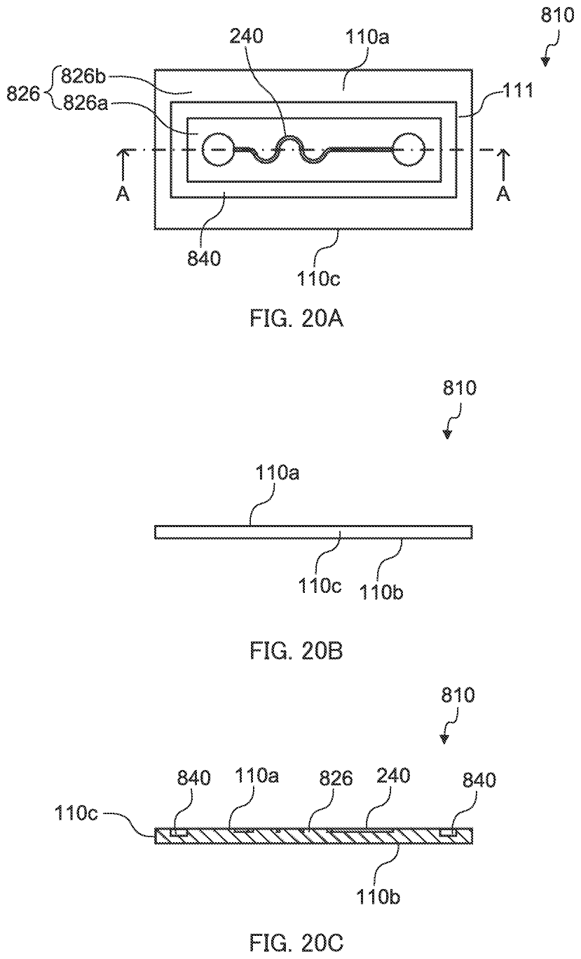

FIGS. 20A to 20C illustrate the configuration of board 810 in fluid handling device 800 according to the modification of embodiment 4 of the present invention. FIG. 20A is a plan view of board 810, FIG. 20B is a side view of board 810, and FIG. 20C is a cross-sectional view taken along line A-A of FIG. 20A.

As illustrated in FIGS. 19A to 19C, and 20A to 20C, board 810 in the present modification is a member that forms the bottom surfaces and the side surfaces of spaces 240 and 840 in fluid handling device 800. Board 810 includes board body 111 and spacer 826.

Spacer 826 includes first spacer 826a and second spacer 826b. First spacer 826a is formed so as to surround a region facing first opening 122a and a region facing third opening 123a. In the present embodiment, space 240 is formed from spacer 826 (first spacer 826a) so as to connect the region facing first opening 122a with the region facing third opening 123a. Second spacer 826b is disposed in such a way that second spacer 826b is closer to the outer edge of first front surface 120a than first spacer 826a is, and separated from first spacer 826a. In space 840 between first spacer 826a and second spacer 826b, adhesive layer 580 is disposed so as to link first spacer 826a with second spacer 826b. Adhesive layer 580 is a part of spacer 826.

Fluid handling device 800 according to the modification of embodiment 4 can be used in the same manner as fluid handling device 100 according to embodiment 1. In addition, fluid handling device 800 according to the modification of embodiment 4 can also be assembled in the same manner as fluid handling device 700 according to embodiment 4.

Effects

As described above, fluid handling devices 700 and 800 according to the present embodiment can provide the same effects as that of fluid handling device 100 according to embodiment 1. In addition, in fluid handling devices 700 and 800 according to the present embodiment, space 840 between first spacer 826a and second spacer 826b is sealed, thereby preventing the leakage of the adhesive to the outside.

### Embodiment 5

Configuration of Fluid Handling Device

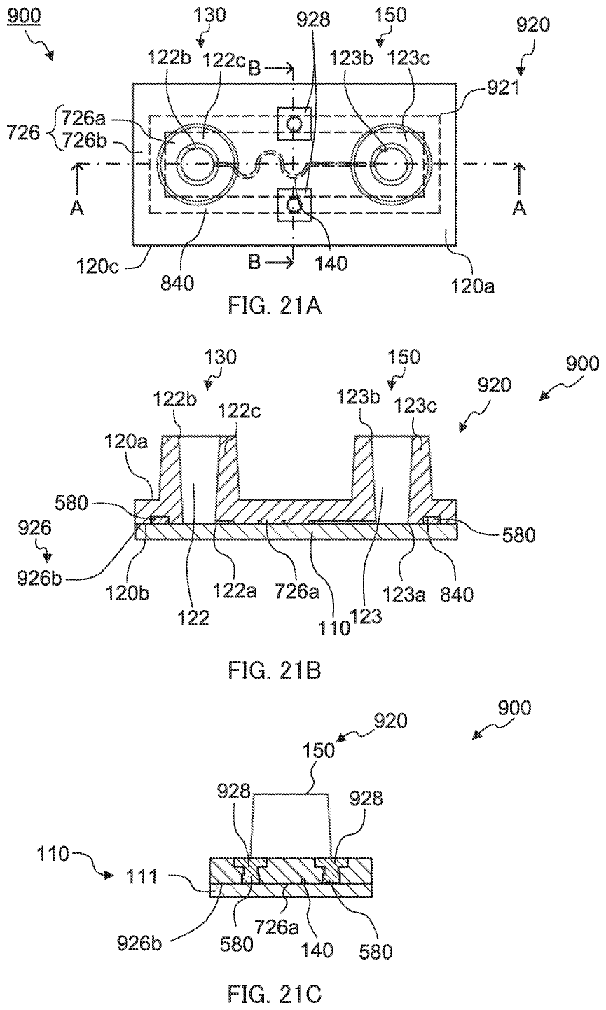

The following describes fluid handling system 900 according to embodiment 5. FIGS. 21A to 21C illustrate the configuration of fluid handling device 900 according to embodiment 5 of the present invention. FIG. 21A is a plan view of fluid handling device 900, FIG. 21B is a cross-sectional view taken along line A-A of FIG. 21A, and FIG. 21C is a cross-sectional view taken along line B-B of FIG. 21A.

As illustrated in FIGS. 21A to 21C, fluid handling device 900 according to embodiment 5 is formed of board 110 and lid 920.

Board 110 is the same as board 110 in embodiment 1, thus the same reference numerals are given and the description thereof will be omitted.

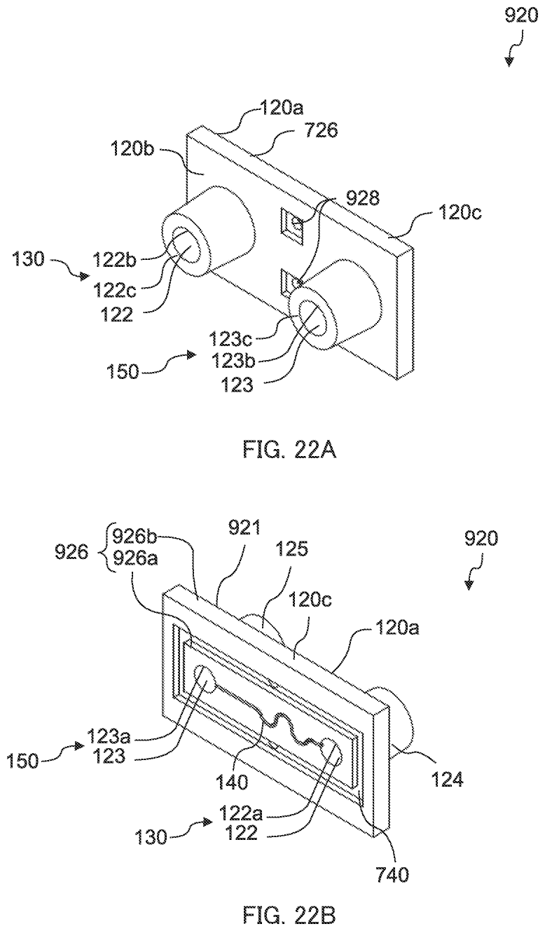

FIGS. 22A and 22B are perspective views illustrating lid 920 in fluid handling device 900 according to embodiment 5 of the present invention. FIG. 22A is a perspective view of lid 920 as viewed from the front side, and FIG. 22B is a perspective view of lid 920 as viewed from the back side.

Lid 920 includes lid body 921, first through hole 122, second through hole 123, spacer 926, at least one third through hole 928, and adhesive layer 580.

For allowing board 110 and lid 920 to adhere to each other, third through hole 928 functions to allow for the injection of an adhesive into the space between first spacer 926a and second spacer 926b. One opening of third through hole 928 opens into space 840 between first spacer 926a and second spacer 926b on second back surface 120b. The other opening of third through hole 928 opens onto a surface other than first surface 121a. In the present embodiment, the other opening of third through hole 928 opens onto second front surface 120a.

Fluid handling device 900 according to the present embodiment is assembled by injecting an adhesive from third through hole 928 with board 110 and lid 920 having been assembled.

Modification

The following describes fluid handling system 1000 according to a modification of embodiment 5. Fluid handling device 1000 according to the modification of embodiment 5 is different from fluid handling device 100 according to embodiment 1 in that board 1010 includes spacer 926 in fluid handling device 1000. The same components as those of fluid handling device 100 according to embodiment 1 are designated by the same reference numerals, and the description thereof will be omitted. In the following, what is different from fluid handling device 100 according to embodiment 1 will be mainly described.

Configuration of Fluid Handling Device

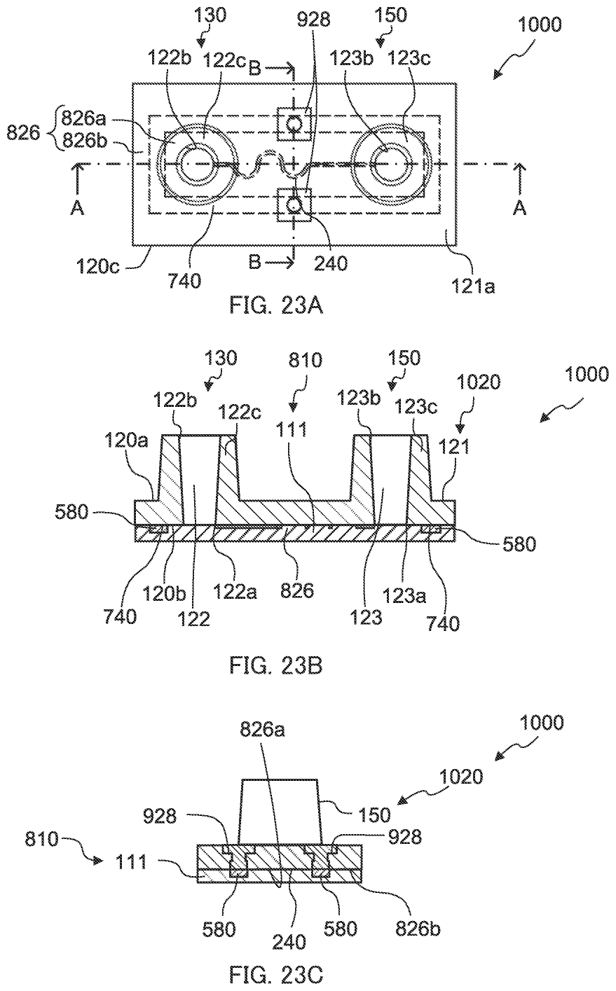

FIGS. 23A to 23C illustrate the configuration of fluid handling device 1000 according to the modification of embodiment 5 of the present invention. FIG. 23A is a plan view of fluid handling device 1000, FIG. 23B is a cross-sectional view taken along line A-A of FIG. 23A, and FIG. 23C is a cross-sectional view taken along line B-B of FIG. 23A.

As illustrated in FIGS. 23A, 23B, and 23C, fluid handling device 1000 according to the modification of embodiment 5 of the present invention is formed of board 810 and lid 1020.

Board 810 is a member that forms the bottom surfaces and the side surfaces of spaces 240 and 840 in fluid handling device 1000. Board 810 includes board body 111 and spacer 826. In other words, board 810 in fluid handling device 1000 according to the present modification is the same as board 810 according to the modification of embodiment 4.

Spacer 826 includes first spacer 826a and second spacer 826b. First spacer 826a is formed so as to surround a region facing first opening 122a and a region facing third opening 123a. In the present embodiment, space 240 is formed from spacer 826 (first spacer 826a) so as to connect the region facing first opening 122a with the region facing third opening 123a. Second spacer 826b is disposed in such a way that second spacer 826b is closer to the outer edge of first front surface 120a than first spacer 826a is, and separated from first spacer 826a. In space 740 between first spacer 826a and second spacer 826b, adhesive layer 580 is disposed so as to link first spacer 826a with second spacer 826b. Adhesive layer 580 is a part of spacer 826.

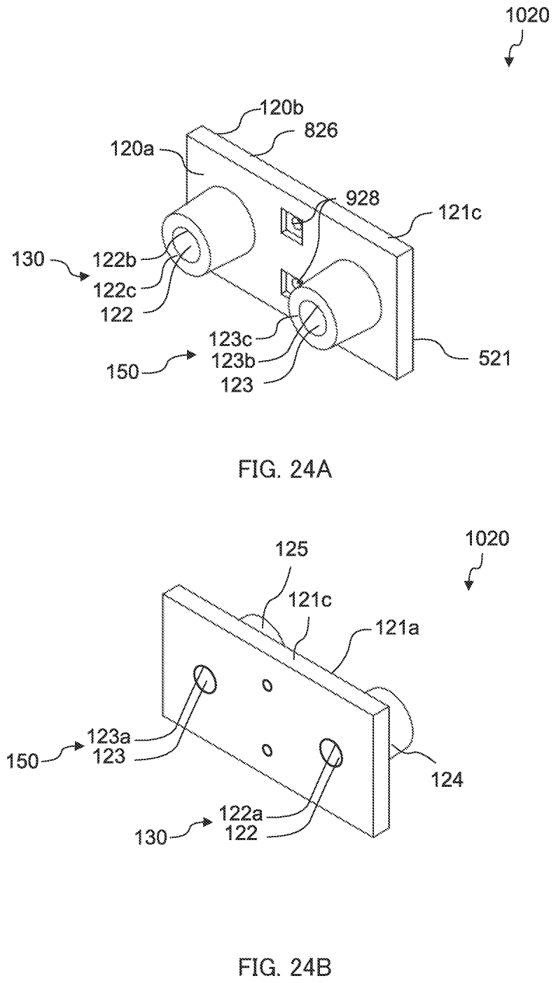

FIGS. 24A and 24B are perspective views illustrating lid 1020 in fluid handling device 1000 according to the modification of embodiment 5 of the present invention. FIG. 24A is a perspective view of lid 1020 as viewed from the front side, and FIG. 24B is a perspective view of lid 1020 as viewed from the back side.

As illustrated in FIGS. 23A, 23B, 23C, 24A, and 24B, lid 1020 is the same as lid 920 in fluid handling device 900 according to embodiment 5, except that lid 1020 does not include spacer 926. In other words, lid 1020 includes lid body 121, first through hole 122, second through hole 123, at least one third through hole 928, and adhesive layer 580.

Fluid handling device 1000 according to the modification of embodiment 5 can be used in the same manner as fluid handling device 100 according to embodiment 1. In addition, fluid handling device 1000 according to the modification of embodiment 5 can also be assembled in the same manner (not illustrated) as fluid handling device 900 according to embodiment 5.

Effects

As described above, fluid handling devices 900 and 1000 according to the present embodiment can provide the same effects as that of fluid handling device 100 according to embodiment 1. In addition, the excess adhesive overflows to the side of inlet 130 and outlet 150 in fluid handling devices 900 and 1000 according to the present embodiment; therefore, the disposition of fluid handling device 1000 in a device where the fluid handling device is to be disposed is not hindered. Further, the adhesive is injected from third through hole 928, thus the operator can work safely and the assembly accuracy of fluid handling devices 900 and 1000 is improved.

### Embodiment 6

Configuration of Fluid Handling Device

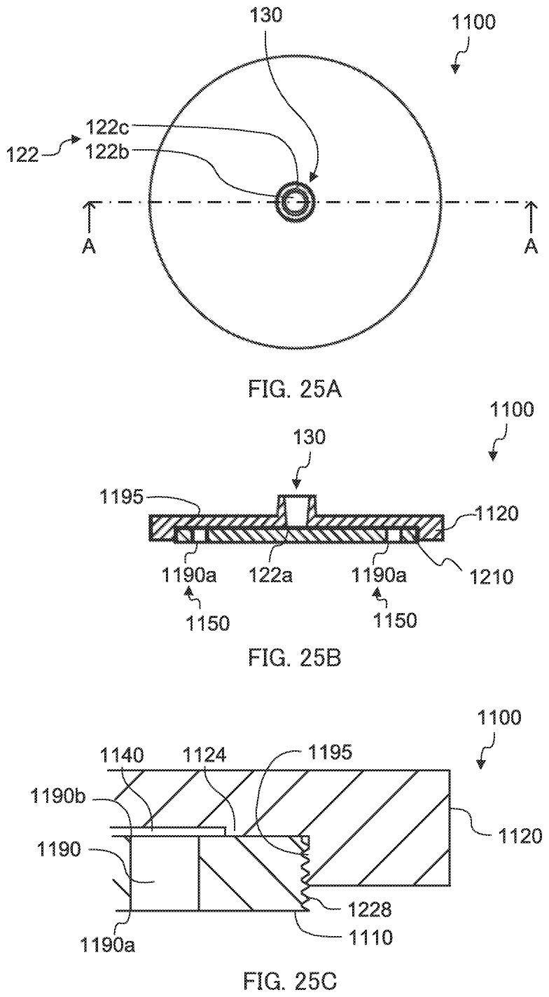

The following describes fluid handling system 1100 according to embodiment 6. FIGS. 25A to 25C illustrate the configuration of fluid handling device 1100 according to embodiment 6 of the present invention. FIG. 25A is a plan view of fluid handling device 1100, FIG. 25B is a cross-sectional view taken along line A-A of FIG. 25A, and FIG. 25C is a partially enlarged cross-sectional view of FIG. 25B.

As illustrated in FIGS. 25A, 25B, and 25C, fluid handling device 1100 according to embodiment 6 is formed of board 1110 and lid 1120. Board 1110 and the lid 1120 are fixed to each other by engaging first screw portion 1195 with second screw portion 1228, which will be described below.

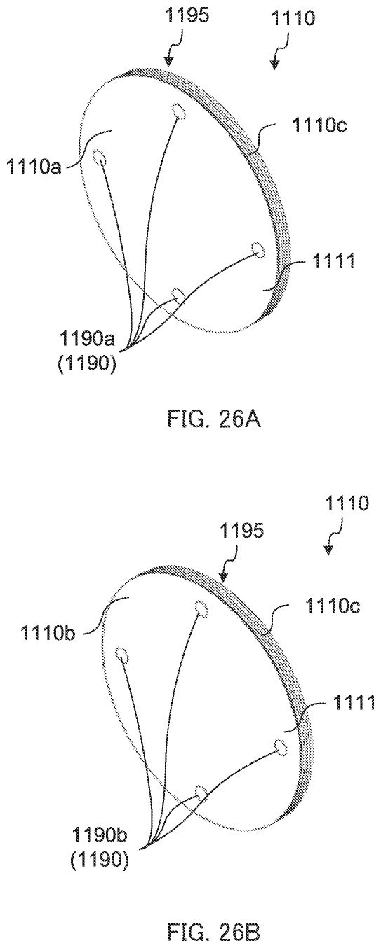

FIGS. 26A and 26B are perspective views illustrating a board in a fluid handling device according to embodiment 6 of the present invention. FIG. 26A is a perspective view of board 1110 as viewed from the front side, and FIG. 26B is a perspective view of board 1110 as viewed from the back side.

As illustrated in FIGS. 25A, 25B, 26A, and 26B, board 1110 is a member that forms the bottom surface of space 1140 in fluid handling device 1100. The shape of board 1110 in plan view is circular. Board 1110 includes board body 1111, at least one fourth through hole 1190, and first screw portion 1195.

The external form of board 1110 is formed of first front surface 1110a disposed to face lid 1120, first back surface 1110b located opposite to first front surface 1110a, and first outer surface 1110c which is a side surface disposed at the outer peripheral portion of first front surface 1110a and first back surface 1110b.

Seventh opening 1190a of fourth through hole 1190 opens onto first front surface 1110a. Eighth opening 1190b of fourth through hole 1190 opens onto first back surface 1110b. First screw portion 1195 is formed on first outer surface 1110c of board 1110.

Fourth through hole 1190 allows for discharge of a fluid from space 1140, and is a part of outlet 1150. Fourth through hole 1290 includes seventh opening 1190a opening on to first front surface 1110a and eighth opening 1190b opening on to first back surface 1110b. Fourth through hole 1190 may have any diameter that allows for the discharge of a fluid. In the present embodiment, the diameter of fourth through hole 1190 is, for example, in the range of 1 to 30 mm.

First screw portion 1195 engages with second screw portion 1128 to fix board 1110 and lid 1120 to each other. In the present embodiment, first screw portion 1195 is a male screw.

Lid 1120 includes lid body 1121, spacer 1126, and guide protrusion 1127.

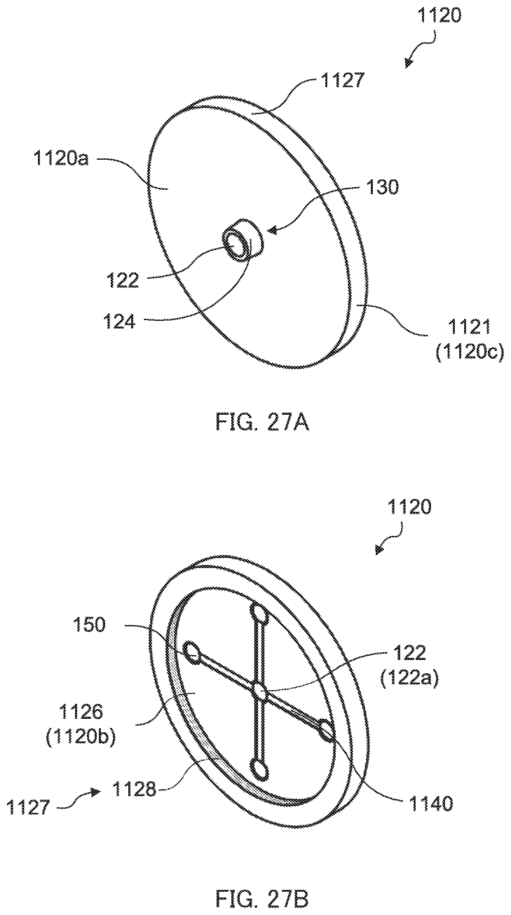

FIGS. 27A and 27B are perspective views illustrating lid 1120 in fluid handling device 1100 according to embodiment 6. FIG. 27A is a perspective view of lid 1120 as viewed from the front side, and FIG. 27B is a perspective view of lid 1120 as viewed from the back side.

As illustrated in FIGS. 27A and 27B, lid 1120 includes lid body 1121, first through hole 122, spacer 1126, and guide protrusion 1127.

Lid body 1121 is formed of second front surface 1120a located opposite to the board 1110 side, second back surface 1120b disposed to face board 1110, and second outer surface 1120c which is a side surface disposed at the outer peripheral portion of second front surface 1120a and second back surface 1120b. In the present embodiment, guide protrusion 1127 is disposed on second back surface 1120b of lid body 1121.

In the present embodiment, the outer shape of lid body 1121 in plan view is circular.

Spacer 1126 is a portion that forms the side surface of space 1140 in fluid handling device 1100. Spacer 1126 is integrally molded with lid body 1121 of lid 1120, and is disposed between first opening 122a and the outer edge of second back surface 1120b, and between seventh openings 1190a of fourth through holes 1190 and the outer edge of second back surface 1120b (in a region inside the outer edge of second back surface 1120b and outside first opening 122a and seventh openings 1190a) in such a way that spacer 1126 is in close contact with board 1110. In the present embodiment, spacer 1226 is also disposed in such a way that space 1140 links first opening 122a with the regions facing seventh openings 1190a. In the present embodiment, spacer 1126 is disposed so that space 1140 has a cross shape.

Guide protrusion 1127 supports board 1110. Second screw portion 1128 is formed on the inner peripheral surface of guide protrusion 1127. Second screw portion 1228 engages with first screw portion 1195 described above. In the present embodiment, second screw portion 1228 is a female screw.

Fluid handling device 1100 according to the present embodiment includes inlet 130, space 1140, and at least one outlet 1150. Inlet 130, space 1140, and outlet 1150 are formed by closely contacting board 1110 with spacer 1126 of lid 1120.

Outlet 1150 functions to allow for the discharge of a fluid in space 1140. Outlet 1150 is a bottomed recess. Outlet 1150 is formed of fourth through hole 1190, lid body 1121, and a part of board 1110.

After a fluid is injected from inlet 130, for example, applying pressure from inlet 130, creating a negative pressure on the outlet 1150 side, or rotating fluid handling device 1100 about inlet 130 allows the fluid of inlet 130 to flow into space 1140. The fluid having flowed into space 1140 is discharged from outlet 1150 to the outside by further applying pressure from inlet 130, creating a negative pressure on the outlet 1150 side, or rotating fluid handling device 1100 about inlet 130.

Fluid handling device 1100 according to the present embodiment is assembled by threadedly engaging first screw portion 1195 with second screw portion 1128.

Modification

The following describes fluid handling system 1200 according to a modification of embodiment 6. Fluid handling device 1200 according to the modification of embodiment 6 is different from fluid handling device 1100 according to embodiment 6 in that board 1210 includes spacer 1226 in fluid handling device 1200. The same components as those of fluid handling device 1100 according to embodiment 6 are designated by the same reference numerals, and the description thereof will be omitted. In the following, what is different from fluid handling device 1100 according to embodiment 6 will be mainly described.

Configuration of Fluid Handling Device

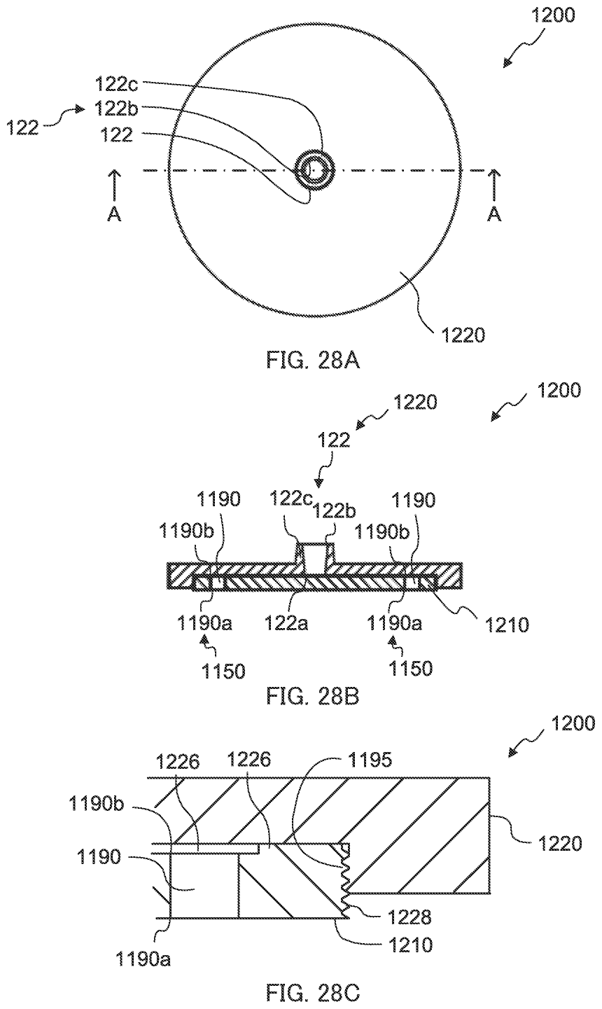

FIGS. 28A to 28C illustrate the configuration of fluid handling device 1200 according to the modification of embodiment 6 of the present invention. FIG. 28A is a plan view of fluid handling device 1200, FIG. 28B is a cross-sectional view taken along line A-A of FIG. 28A, and FIG. 28C is a partially enlarged cross-sectional view of FIG. 28B.

As illustrated in FIGS. 28A and 28B, fluid handling device 1200 according to the modification of the present embodiment is formed of board 1210 and lid 1220.

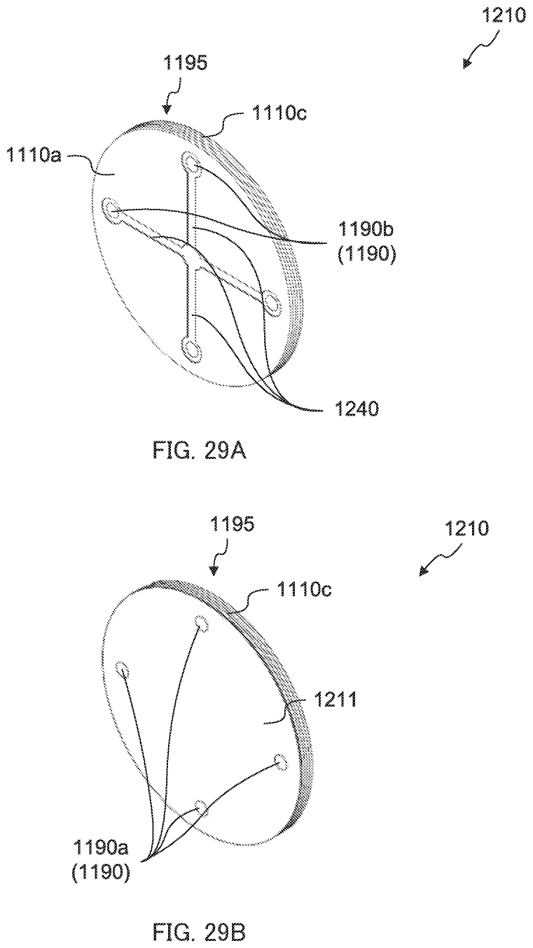

FIGS. 29A and 29B are perspective views illustrating board 1210 in fluid handling device 1200 according to the modification of embodiment 6 of the present invention. FIG. 29A is a perspective view of board 1210 as viewed from the front side, and FIG. 29B is a perspective view of board 1210 as viewed from the back side.

In the present modification, board 1210 is a member that forms the bottom surface and the side surface of space 1240 in fluid handling device 1200. Board 1210 includes board body 1211 and spacer 1226. Spacer 1226 is integrally molded with board body 1211 of board 1210, and is disposed between the outer edge of first front surface 1110a and a region facing first opening 122a, and between the outer edge of first front surface 1110a and a region facing eighth openings 1190b in such a way that spacer 1226 is in close contact with lid 1220. In the present modification, spacer 1226 is also disposed in such a way that space 1240 links the region facing first opening 122a with eighth openings 1190b.

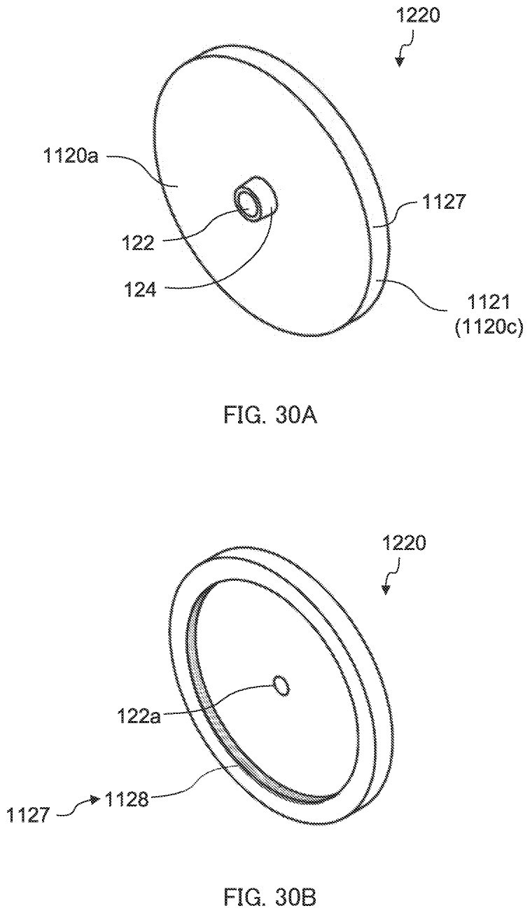

FIGS. 30A and 30B are perspective views illustrating lid 1220 in fluid handling device 1200 according to the modification of embodiment 6. FIG. 30A is a perspective view of lid 1220 as viewed from the front side, and FIG. 30B is a perspective view of lid 1220 as viewed from the back side.

As illustrated in FIGS. 30A and 30B, lid 1220 includes lid body 1121, first through hole 122, first cylinder portion 124, and guide protrusion 1127. In other words, lid 1220 is the same as lid 1120 in fluid handling device 1100 according to embodiment 6, except that lid 1220 does not include spacer 1126.

In addition, fluid handling device 1200 according to the modification of embodiment 6 can also be assembled in the same manner (not illustrated) as fluid handling device 1100 according to embodiment 6.

Effects

As described above, fluid handling devices 1100 and 1200 according to the present embodiment can be readily assembled because no adhesive is used.

### REFERENCE SIGNS LIST

100, 200, 300, 400, 500, 600, 700, 800, 900, 1000, 1100, 1200 Fluid handling device
110, 210, 610, 810, 1010, 1210 Board
110a, 1110a First front surface
110b, 1110b First back surface
110c, 1110c First outer surface
111, 1111, 1211 Board body
120, 220, 320, 420, 520, 720, 920, 1020, 1120, 1220 Lid
120a, 1120a Second front surface
120b, 1120b First back surface
120c, 1120c Second outer surface
121, 921, 1121 Lid body
121a First surface
122 First through hole
122a First opening
122b Second opening
123 Second through hole
123a Third opening
123b Fourth opening
124 First cylinder portion
125 Second cylinder portion
126, 226, 526, 626, 726, 826, 926, 1126, 1226 Spacer
127 Claw portion
127a Fixing portion
127b Guide portion
127c Holding portion
130 Inlet
150, 1150 Outlet
360 Magnetic member
361 Central portion
362 Frame portion
363 Pressing portion
370 Facing member
580 Adhesive layer
726a, 826a First spacer
726b, 826b Second spacer
928 Third through hole
1127 Guide protrusion
1128 Second screw portion
1190 Fourth through hole
1190a Seventh opening
1190b Eighth opening
1195 First screw portion
1223a Third opening
1228 Second screw portion
1290 Fourth through hole

1. A fluid handling device, comprising:
a board that includes a board body including a first surface; and
a lid that faces the first surface of the board and is disposed so as to form a space between the board and the lid,
wherein the lid includes
a lid body including a second surface that faces the board, and
a first through hole that allows for injection of a fluid into the space, the first through hole including a first opening that opens onto the second surface and a second opening that opens onto a surface other than the second surface, wherein
the lid further includes a spacer integrally molded with the lid body, the spacer being disposed between the first opening and an outer edge of the second surface in such a way that the spacer is in close contact with the board, or
the board includes a spacer integrally molded with the board body, the spacer being disposed between a region facing the first opening and an outer edge of the first surface in such a way that the spacer is in close contact with the lid.

2. The fluid handling device according to claim 1, wherein:
the lid further includes a second through hole that allows for discharge of the fluid from the space, the second through hole including a third opening that opens onto the second surface and a fourth opening that opens onto a surface other than the second surface; and
the spacer is integrally molded with the lid body, and is disposed between the first opening and the outer edge of the second surface and between the third opening and the outer edge of the second surface in such a way that the spacer is in close contact with the board.

3. The fluid handling device according to claim 1, wherein:
the lid further includes a second through hole that allows for discharge of the fluid from the space, the second through hole including a third opening that opens onto the second surface and a fourth opening that opens onto a surface other than the second surface; and
the spacer is integrally molded with the board body, and is disposed between the region facing the first opening and the outer edge of the first surface and between a region facing the third opening and the outer edge of the first surface in such a way that the spacer is in close contact with the lid.

4. The fluid handling device according to claim 1, wherein the lid further includes a claw portion for holding the board by engaging with the board.

5. The fluid handling device according to claim 1, further comprising:
a magnetic member for bringing the lid and the board into close contact with each other by a magnetic force, the magnetic member being disposed with respect to the lid to be on a side opposite to the board, the magnetic member containing a magnet or a ferromagnet.

6. The fluid handling device according to claim 1, wherein:
the spacer is integrally molded with the lid body;
the spacer forms a space opening onto an outer edge of the lid; and
an adhesive layer is disposed in the space opening onto the outer edge of the lid.

7. The fluid handling device according to claim 1, wherein:
the spacer is integrally molded with the board body;
the spacer forms a space opening onto an outer edge of the board; and
an adhesive layer is disposed in the space opening onto the outer edge of the board.

8. The fluid handling device according to claim 1, wherein:
the spacer includes a first spacer that is integrally molded with the lid body and disposed on a side of the first opening, and a second spacer disposed in such a way that the second spacer is closer to the outer edge of the second surface than the first spacer is and separated from the first spacer; and
in a space between the first spacer and the second spacer, an adhesive layer is disposed so as to link the first spacer with the second spacer.

9. The fluid handling device according to claim 1, wherein:
the spacer includes a first spacer that is integrally molded with the board body and disposed on a side of the region facing the first opening, and a second spacer disposed in such a way that the second spacer is closer to the outer edge of the first surface than the first spacer is and separated from the first spacer; and
in a space between the first spacer and the second spacer, an adhesive layer is disposed so as to link the first spacer with the second spacer.

10. The fluid handling device according to claim 8, wherein:
the lid further includes a third through hole that allows for injection of an adhesive into the space between the first spacer and the second spacer, the third through hole opening onto the space between the first spacer and the second spacer and onto a surface other than the first surface.

11. The fluid handling device according to claim 1, wherein:
the lid further includes a first screw portion; and
the board further includes a second screw portion to be threadedly engaged with the first screw portion.
